(19) United States
(12) Patent Application Publication
(10) Pub. No.: US 2023/0092232 A1
(43) Pub. Date: Mar. 23, 2023

(54) COMPOUNDS THAT MODULATE ANTI-TUMOR IMMUNITY AND METHODS OF DOING THE SAME

(71) Applicant: University of Florida Research Foundation, Incorporated, Gainesville, FL (US)

(72) Inventors: David A. Ostrov, Gainesville, FL (US); Christopher R. Cogle, Gainesville, FL (US)

(73) Assignee: University of Florida Research Foundation, Incorporated, Gainesville, FL (US)

(21) Appl. No.: 17/802,872

(22) PCT Filed: Feb. 26, 2021
(86) PCT No.: PCT/US2021/020109

§ 371 Date: Aug. 26, 2022

Related U.S. Application Data
(60) Provisional application No. 63/125,118, filed on Dec. 14, 2020.
(60) Provisional application No. 62/983,538, filed on Feb. 28, 2020.

(51) Int. Cl. A61K 31/52; A61K 9/00; A61K 31/519; A61K 31/133; A61K 31/047; A61P 35/02; A61K 45/06

(52) U.S. Cl. CPC A61K 31/52; A61K 9/0053; A61K 31/519; A61K 31/133; A61K 31/047; A61P 35/02; A61K 45/06

(57) ABSTRACT

Provided herein are compositions and methods for eliciting a desired immune response in a subject in need thereof. The compositions and methods are particularly useful as anti-cancer immune therapy by exploiting a subject's propensity for drug (e.g small molecule) hypersensitivity. Of particular significance is the application in personalized immune therapy for cancer patients utilizing or repurposing existing FDA approved drugs.

### BACKGROUND

The use of immunotherapy for use in treating patients with cancer requires the in vivo enhancement of peripheral blood mononuclear cell (PBMC) response, including lymphocytic response, through the activation and stimulation of the human leukocyte antigen (HLA) system. However, there are currently 26,512 identified HLA and related alleles, and only a small subset of HLA molecules have demonstrated associations with drug-induced enhancement of T cell activity. Thus, implementing HLA- and HLA allele-specific therapies useful for treating cancer through immunotherapy has been difficult.

### SUMMARY

There is an urgent need to develop novel therapeutic strategies to selectively enhance antitumor activity by targeted augmentation of impaired CD8⁺ T cell responses in patients with cancer. In some aspects, methods disclosed herein involve a three dimensional analysis of HLA molecules to identify one or more binding sites for small molecules. In some aspects, methods disclosed herein are useful to identify one or more small molecules that that bind to an HLA molecule. In some embodiments, one or more small molecules can be used to activate an HLA molecule. In some embodiments, one or more small molecules enhance peptide/HLA interactions. In some embodiments, one or more small molecules selectively bind to and activate particular HLA allelic variants. In some embodiments, one or more small molecules selectively binds to one or more selective MHC alleles and is capable of eliciting an immune hypersensitivity reaction, in a subject. In some embodiments, the one or more small molecules is an active pharmaceutical ingredient (API) in an approved drug product (e.g., FDA-approved or approved by other regulatory agency), a prodrug, a metabolite thereof, or a drug-like small molecule.

In some embodiments, the one or more small molecules is a drug that was previously approved for a different indication, such as a non-cancer indication. As contemplated herein, a small molecule or HLA-binding molecule of the present disclosure, which in some embodiments is useful to treat cancer, is not an FDA-approved anti-cancer treatment, is not a chemotherapy or chemotherapeutic agent, and/or is not known by those skilled in the art to be an anti-cancer agent or treatment.

Use of FDA approved drugs (e.g., small molecules) in such a personalized strategy can be advantageous due to the known toxicities of these compounds, allowing for rapid implementation of treatment strategies and the tailoring of binding specificity to reduce risk of side effects.

In some embodiments, one or more small molecules are useful to treat a disease (e.g., a cancer). In some embodiments, one or more small molecules are useful for a personalized therapy to treat a disease (e.g., a cancer) in a subject who expresses particular HLA allelic variant(s). Accordingly, provided herein are methods of treatment for subjects suffering from, or diagnosed with, a cancer. Provided herein are methods of treatment for subjects in one or more subpopulations that express a particular HLA allelic variant. In some embodiments, provided herein are methods of treatment for subjects, such as human subjects, who express an HLA-B*57:01⁺ variant. The disclosed methods may prevent or slow the development, progression, and/or spread of a cancer in the subject, and/or ameliorate one or more symptoms associated with the cancer.

Also provided herein are methods for augmenting an anti-cancer or anti-tumor immune response, in a subject in need thereof. In some aspects, the subject suffers from, or is diagnosed with, cancer. In some aspects, the augmented anti-cancer or anti-tumor immune response is associated with or evidenced by augmented CD8⁺ T cell responses and/or a severe inflammatory response, including but not limited to an immune hypersensitivity response (e.g., an immune hypersensitivity reaction).

As used herein, the terms “immune hypersensitivity reaction” and “immune hypersensitivity response” are used interchangeably. In some embodiments, an immune hypersensitivity reaction occurs when a drug (including but not limited to an approved drug), triggers an overreaction of the immune system that would otherwise not be desirable for the original intended use of the drug. In some embodiments, however, an immune hypersensitivity reaction may be clinically useful in other indications, such as, for example in inducing an anti-tumor immune response. Symptoms of immune hypersensitivity reaction include at least two of the following: fever, rash, gastrointestinal symptoms (e.g., nausea, vomiting, abdominal pain), fatigue, cough, and/or dyspnea to a degree, which would evidence an overaction of the immune response respective to the drug being investigated.

The current state of the art in clinical practice may dissuade physicians from continuing administration of a drug (e.g., abacavir) after presentation of a symptom (or a second symptom) associated with an immune hypersensitivity presents in a patient or subject. For instance, clinical practice may dissuade physicians from administering a second, third, or fourth dose of a drug after presentation of a symptom (or a second symptom) associated with an immune hypersensitivity presents in a patient or subject. However, the typical signs and symptoms of an immune hypersensitivity reaction may nevertheless, in many instances, be tolerable to patients and subjects, particularly those patients experiencing clinical signs or symptoms of a disease such as cancer. Symptoms of an immune hypersensitivity reaction may or may not increase in severity over time if administration of the drug is continued despite the development of immune hypersensitivity reaction symptoms. In instances where such symptoms are severe from the first administration of the drug and/or increase in severity over time with continued administration of the drug, a point of intolerance may be reached. The dosage at which a point of intolerance is reached is unique to each patient, and may be considered a patient's maximum tolerable dose (MTD) of the drug. Clinicians practicing the methods of the present invention may or should monitor patients for severe immune hypersensitivity reactions for the some or the entire duration of treatment. Thereafter, physicians should discontinue or augment treatment immediately if or when a point of intolerance is reached for a patient or subject.

In some embodiments, the methods for augmenting an anti-cancer or anti-tumor immune response comprise administering a composition comprising a therapeutically effective amount of a small molecule to the subject. In some embodiments, the small molecule preferentially binds to one or more selective MHC alleles and is capable of eliciting an immune hypersensitivity reaction in the subject. In some embodiments, such elicitation of an immune hypersensitivity response augments said anti-cancer or anti-tumor immune response. In some embodiments, the small molecule preferentially binds to one or more selective HLA and elicits the immune hypersensitivity reaction in the subject, thereby augmenting said anti-cancer or anti-tumor immune response.

In some embodiments, the step of administering takes place in conjunction with another therapy. In some embodiments, another therapy may comprise a cancer therapy, such as, for example, a chemotherapy or other standard-of-care cancer therapy. Chemotherapeutic agents and standard-of-care cancer therapies are known in the art, and any cancer therapy may comprise the secondary therapy as contemplated herein.

In some embodiments, the methods of treatment comprise augmentation of T cell responses by inducement of a hypersensitivity response in a subject to one or more small molecules, following administration of the small molecule(s) to the subject. Accordingly, provided herein are methods of treatment comprising the step of eliciting a hypersensitivity response to one or more small molecules, such as a small molecule that selectively binds to and activates particular HLA allelic variants. In some embodiments, such small molecules are APIs of one or more FDA-approved drug products. In some embodiments, the small molecule is an API of an FDA-approved product that is contraindicated for subjects that expresses a particular HLA allelic variant, such as the HLA-B*57:01⁺ variant. In particular embodiments, the small molecule comprises abacavir.

Accordingly, in some embodiments, the disclosed methods comprise the step of eliciting a hypersensitivity response to abacavir. The disclosed methods may involve elicitation of this response in a subject, or a subpopulation of subjects, that expresses a particular HLA allelic variant, such as the HLA-B*57:01⁺ variant. The hypersensitivity response may be induced by administering a first dose of the small molecule (e.g., abacavir) and administering a second or additional dose. Additional doses may be administered to enhance the hypersensitivity response to a level effective to treat or ameliorate a cancer, or the underlying symptoms thereof. Additional doses may be administered that are below, or exceed, a level of discomfort for the subject (e.g., a human subject). In some embodiments, the disclosed methods comprise methods of treatment of a subject suffering from or diagnosed with cancer comprising i) administering a first dose of abacavir sufficient to induce an immune response, e.g., an immune hypersensitivity reaction, in the subject, and ii) administering a second or subsequent dose of abacavir. In some embodiments, the disclosed methods comprise administering a third, fourth, fifth, or subsequent dose of abacavir to the subject. In certain embodiments, the methods comprise administering one or more doses to a subject that expresses the HLA-B*57:01⁺ variant.

Some aspects therefore contemplate a method comprising (i) administering orally a first dose of abacavir to a subject suffering from or diagnosed with cancer, and (ii) administering orally a second dose of abacavir to the subject. In some embodiments, the subject has an HLA-B*57:01 genotype (e.g., expresses the HLA-B*57:01⁺ variant). In some embodiments, the abacavir is administered according to the methods described in Example 3 and/or Table 3.

In some embodiments, the one or more small molecules comprises a newly discovered drug (de novo drug discovery), e.g., a small molecule. The newly discovered drug may selectively bind to and activate particular HLA allelic variants, including but not limited to the HLA-B*57:01⁺ variant.

In some embodiments, the three-dimensional characterization involves in silico modeling, which is used to identify structural features of the HLA molecule that are favorable for facilitating HLA binding to drug-like small molecules. In some embodiments, the structural features of the HLA molecule that are favorable for binding to drug-like small molecules comprise one of several criteria used to facilitate the selection of candidate compounds that would be expected, based on known binding affinities of the compounds, to effectively bind the targeted HLA molecule.

In some embodiments, candidate compounds are further evaluated in vitro, for example in a cell-based assay (e.g., using cells taken from a subject, for example a subject known to express an HLA molecule of interest), or an animal model (e.g., an animal model of a disease such as cancer) to evaluate HLA binding, immune stimulation, and/or disease response.

In some embodiments, one or more candidate compounds predicted to bind an HLA molecule of interest (and/or shown to be effective in a cell-based and/or animal model) are administered to a subject (e.g., a subject having a disease, for example cancer), for example in an amount that is effective to assist in the treatment of a disease (e.g., a cancer) in, e.g., a human subject.

In some embodiments, one or more compounds bind an HLA molecule of interest and stimulate an immune response, e.g., an immune hypersensitivity reaction, in a subject. In some embodiments, the stimulated immune response enhances CD8⁺ T cell mediated adaptive immunity. Thus, in some embodiments, the administration of one or more candidate compounds can be useful in the treatment of disease (e.g., cancer) through the stimulation of an immune response (e.g., through CD8⁺ T cell mediated adaptive immunity).

Accordingly, methods and compounds described in this disclosure can be useful in a personalized treatment for cancer in a subject (e.g., in a human cancer patient) by enhancing CD8⁺ T cell mediated adaptive immunity through the targeted binding of specific sites on specific HLA molecules (e.g., on one or more HLA alleles expressed in the subject). In some embodiments, the methods described herein are particularly useful for subjects or patients that have shown resistance to standard-of-care treatments for cancer.

In practicing any of the methods disclosed herein, the subject administered with a subject small molecule may exhibit a propensity for immune hypersensitivity, elicitable by the small molecule. By “propensity” it is meant that the subject possesses an allele of a gene (e.g., an allelic variant form of a gene, such as HLA-B*57:01, i.e., an MHC) that interacts with certain compounds (e.g., abacavir) such that when said subject is administered said compound, a hypersensitivity reaction will be induced at a rate of incidence that is higher than that observed in subjects who do not possess said allele and are administered the same compound. Where desired, such propensity for immune hypersensitivity is ascertained by testing for the presence of the one or more selective MHC expressed in the subject to which the small molecule preferentially binds. Accordingly, the methods may include a step of testing the subject for the presence of an expressed target MHC or HLA prior to administering to the subject a small molecule to elicit the desired anti-cancer immune response.

In some embodiments, one or more HLA binding molecules can be administered to a subject in combination with one or more additional immune stimulating molecule(s) and/or additional cancer therapeutic molecule(s). In some embodiments, one or more HLA binding molecules can be administered to a subject along with a cancer antigen (e.g., a cancer-associated neoantigen and/or an antigen that is overexpressed in cancer, for example a patient-specific cancer antigen). In some embodiments, HLA binding molecule(s) are administered together with additional molecule(s). In some embodiments, the HLA binding molecule(s) and additional molecule(s) are provided together in the same composition. In some embodiments, the HLA binding molecule(s) and additional molecule(s) are provided in separate compositions and administered together. However, in some embodiments, HLA binding molecule(s) are administered at different times and/or at different frequencies than the additional molecule(s).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows HLA supertype-specific sites in the antigen binding cleft of HLA-A2 with structural features favorable for binding to drug-like small molecules.

FIG. 2 shows sites for drug occupancy within the B and F pockets of HLA-A2 to alter peptide binding and the formation of neoantigen peptide/HLA complexes.

FIGS. 3A and 3B show the atomic coordinates of the HLA-A2 B and/or F pockets which were used as the basis for in silico selection of candidate compounds. FIG. 3A shows both B and F pockets.

FIG. 3B shows an enlarged view of the B pocked with a high ranked compound docked in the B pocket.

FIGS. 4A-4I show the chemical structures of compounds selected to bind HLA-A2.

FIG. 5 shows drugs predicted to bind HLA-A2 that were tested using PBMC from HLA typed normal individuals.

FIGS. 6A-6L show the chemical structures of compounds selected to bind HLA-DR3.

### DETAILED DESCRIPTION

In some aspects, methods contemplated herein involve screening numerous known compounds and drug-like small molecules (for example 139,735) to identify those compounds most likely to bind to, or otherwise stimulate an immune response from, HLA molecules. In some embodiments, those HLA molecules are pre-selected based on a known affiliation with a disease state. In some embodiments, the identified compounds activate HLA molecules generally, rather than modulating a specific peptide HLA interaction. Thus, in some embodiments, compounds identified using methods described herein enhance the peptide loading without altering the binding affinity between the compound and the HLA molecule of interest. In some embodiments, the compounds identified using the methods described herein bind the HLA molecule outside the peptide binding cleft. In some embodiments, enhanced interaction between the compound and the HLA molecule of interest involves increased binding affinity between the compounds and HLA molecule (for example abacavir and HLA-A2).

In some aspects, the disclosure provides methods of eliciting a hypersensitivity reaction to a small molecule that binds an HLA molecule of interest, and harnessing that hypersensitivity reaction to treat a disease, disorder, or condition. In some embodiments, the disease, disorder or condition is cancer. In certain aspects, the small molecule is approved or indicated to treat a disease or disorder other than cancer, such as HIV/AIDS. In some embodiments, the small molecule is a nucleoside analog reverse-transcriptase inhibitor (NRTI). In certain embodiments, the small molecule is abacavir.

The desired hypersensitivity reaction represents a stimulation of an immune response (e.g., an immune hypersensitivity reaction), and in particular, enhanced CD8⁺ T cell mediated adaptive immunity. Accordingly, in several embodiments, the hypersensitivity reaction elicited by any of the disclosed methods is measured as an elevation in T cell count. The T cell count may be quantified by various methods including but not limited to quantitative flow cytometry of CD8+, CD4+, or CD3+ cells in the peripheral blood or bone marrow. In some embodiments, the hypersensitivity reaction is measured using any other method known in the art, such as white blood cell (WBC) count, absolute lymphocyte count (ALC), B cell count, macrophage count, dendritic cell count, or PBMC count. As used herein, the term “sufficient to induce an immune response” refers to an elevation in T cell count in the subject above the subject's baseline T cell count. The baseline T cell count may be measured immediately before abacavir treatment, one day before treatment, 2-4 days before treatment, 5 days to a week before treatment, or more than a week before treatment. The T cell count may be measured at various time points. In some embodiments, the T cell count is measured at 1 month, 3 months, 6 months, 9 months, and/or 9 months.

In some aspects, the disclosure provides personalized therapies for one or more patients (e.g., subjects), such as for eliciting hypersensitivity reactions based on the HLA subtype of the subpopulation of patient, such as an HLA-B subtype. In some embodiments, the subpopulation of patients expressed the HLA-B*57:01 subtype. The subpopulation of patients may comprise patients suffering from, or diagnosed with, cancer, such as liver cancer, lung cancer, a blood cancer (such as a leukemia), head and neck cancer, colorectal cancer, pancreatic cancer, oral cancer, and other cancers. In some embodiments, the patient is treated with abacavir. In some embodiments, the treatment of a patient with abacavir comprises administering abacavir to the patient. In some embodiments, the administration of abacavir to the patient is according to the methods described in Example 3 and Table 3.

In some aspects, the disclosure provides for the de novo discovery and screening of drugs (e.g., small molecules) that provide a stimulation of an immune response, and in particular, enhanced CD8⁺ T cell mediated adaptive immunity. These methods may comprise a step of in silico modeling and in vitro testing. These methods may further comprise validation of candidates scored as “hits” following in vitro testing in in vivo models, such as animal subjects (e.g., rodent subjects). Accordingly, provided herein are methods for screening candidate small molecules that activate particular HLA allelic variants comprising a step of in silico modeling and further a step of in vitro evaluation. The in vitro evaluation step may comprise evaluations of T cell stimulation (e.g., measurement of T cell counts) and killing of cancer cells. In some embodiments, the candidate small molecules are screened against a liver (hepatocellular) cancer cell line.

Accordingly, in some embodiments, the disclosure provides methods of identifying a compound that enhances T cell mediated immunity by HLA binding, the method comprising a step of (a) performing a structure-based analysis to identify a compound that binds to an HLA molecule, and (b) evaluating the identified compound using a cell-based assay and/or an animal model to determine whether the compound enhances T cell mediated immunity. In some embodiments, the step of performing a structure-based analysis comprises a step of modeling in silico the structure of the HLA allelic variant. In some embodiments, the step of modeling in silico is conducted on a computer-based structural design program such as DOCK6 (UCSF).

### Compounds

In some embodiments, a compound is administered to a subject. As used herein, a compound can be a small molecule capable of eliciting a desired immune response, e.g., an immune hypersensitivity reaction, when administered into a subject in need thereof. In some embodiments, the small molecule preferentially or selectively binds to a given MHC or HLA in a human subject to elicit an immune activity of T cells.

A preferential or selective binding of a subject small molecule to a given MHC allele or HLA can be demonstrated by any of the methods known in the art or disclosed herein. In an embodiment, an in silico assay is performed, which utilizes a surface plasmon resonance (SPR) pMHC stability assay that detects changes in mass at the surface of a gold plated sensor chip. This technology enables the determination of pMHCI half-life by detecting protein density at the sensor chip surface in real time between a subject small molecule and a target HLA.

Where desired, a computer-assisted analysis is carried out to establish preferential binding of a small molecule to a target MHC or HLA. Non-limiting examples include Tsites program (see, e.g., Rothbard and Taylor, EMBO J. 7:93-100, 1988; Deavin et al., Mol. Immunol. 33:145-155, 1996), which searches for motifs expressed by a subject small molecule that have the potential to elicit responses by cells expressing the target MHC or HLA.

In another embodiment, a direct binding assay is utilized. A direct binding assay can measure the ability of a small molecule to stabilize the HLA or MHC-peptide complex, which will keep its native conformation if the binding affinity of tested peptide is high enough. A known T cell epitope can be used as a positive control, and each small molecule may be given a score by testing versus the positive control peptide.

In yet another embodiment, a competition binding assay can be utilized. A competition binding assay utilizes a subject small molecule to assay for its ability to compete against labeled high-affinity control molecules, such as peptides, for binding to HLA or MHC molecules. IC50 data is calculated by analyzing the dose-response curve.

In another embodiment, a real-time kinetic binding assay is employed. This assay can give kinetic information about the on- and off-rate at which each small molecule interacts with HLA or MHC molecules in real-time. It can provide complete information as to whether a peptide could be presented for long enough to be a suitable binding molecule. For example, HLA binding molecules with fast on- and off-rates may not be suitable candidates. Protocols based on fluorescence polarization or surface plasmon resonance (SPR) can be employed.

Cell-based assays can also be employed to test for a small molecule's ability to preferentially or selectively bind to a given HLA or MHC allele, and for its ability to elicit an immune cell response. For example, an HLA binding assay can be carried out either using cells which express high numbers of empty (unoccupied) HLA molecules (e.g., cellular binding assay), or using purified HLA molecules. Subject HLA binding molecules can be tested for their capacity to induce a CTL response in naive subjects, either in vitro using human or non-human lymphocytes, or in vivo using HLA-transgenic animals. To further confirm immunogenicity, a peptide may be tested using an HLA A2 transgenic mouse model and/or any of a variety of in vitro stimulation assays.

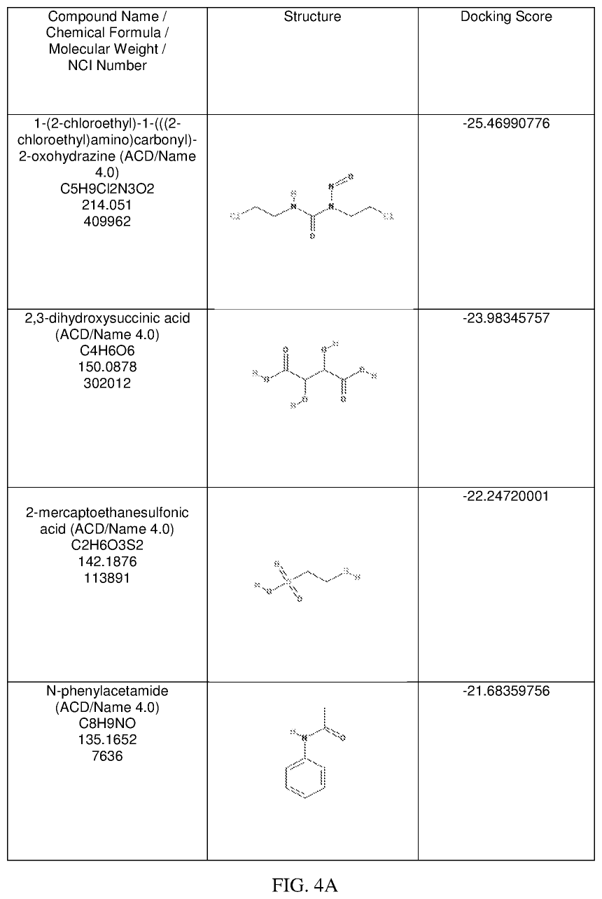

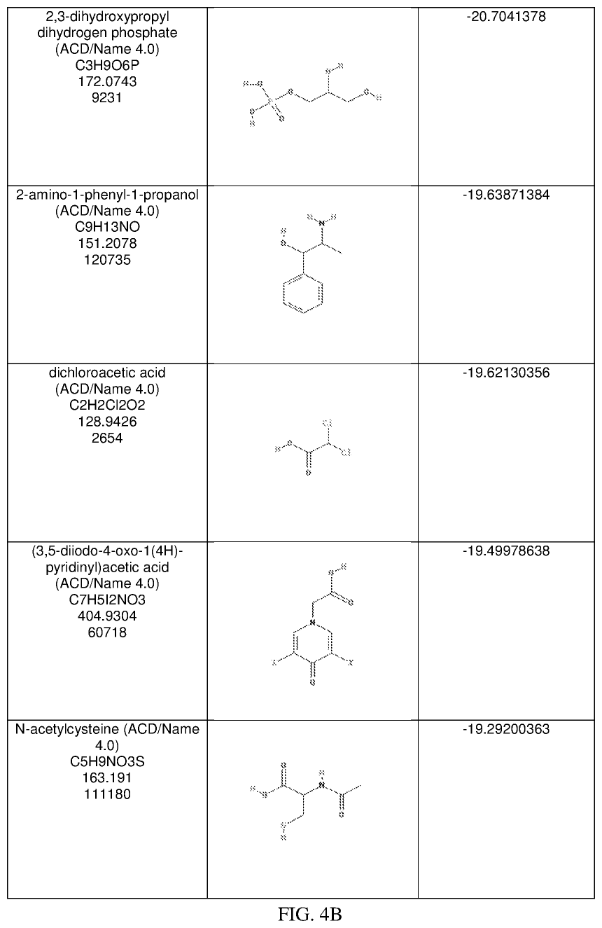

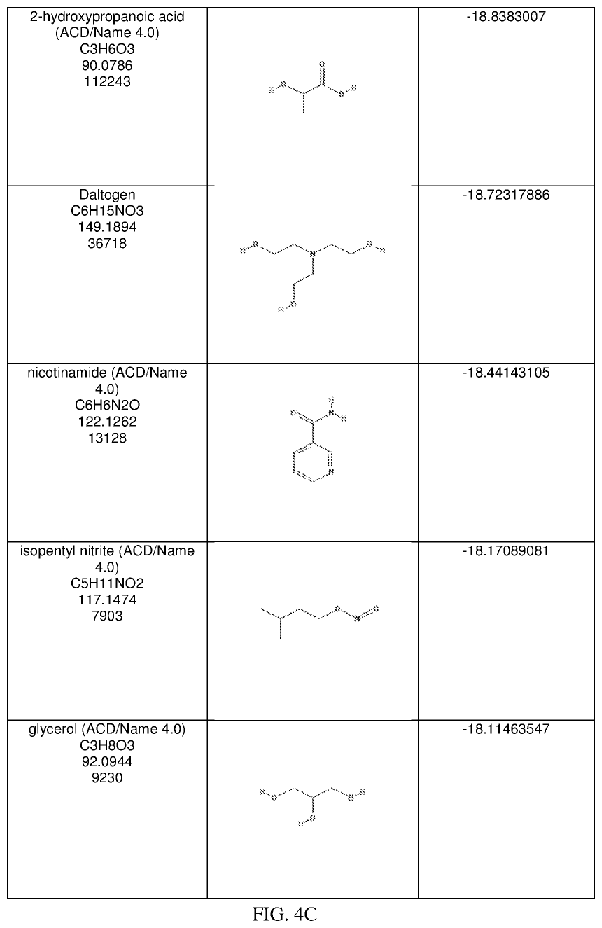

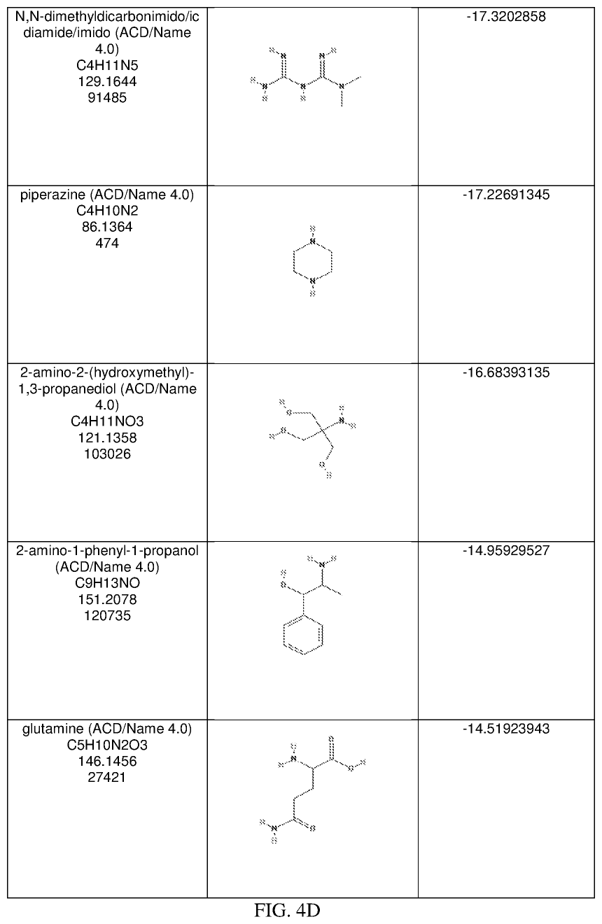

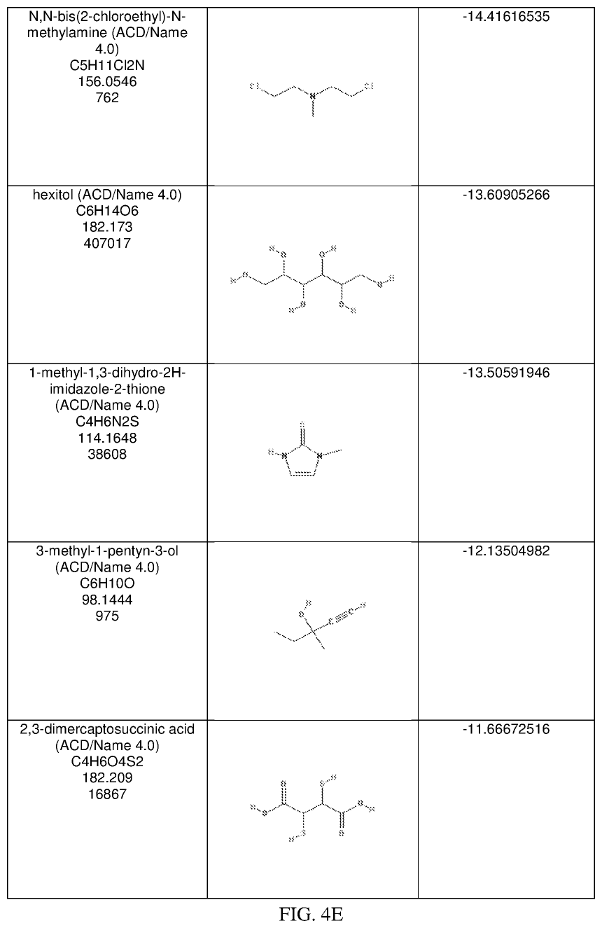

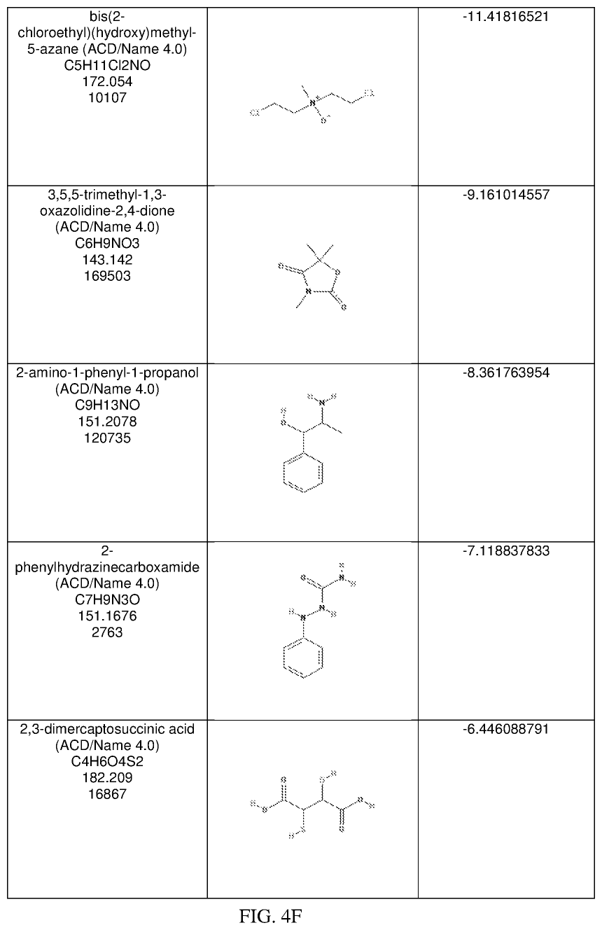

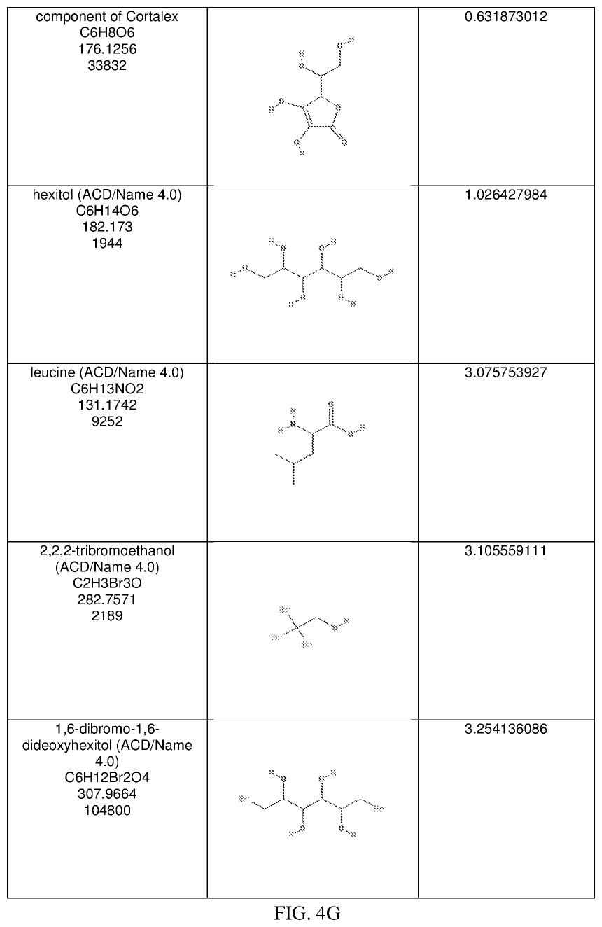

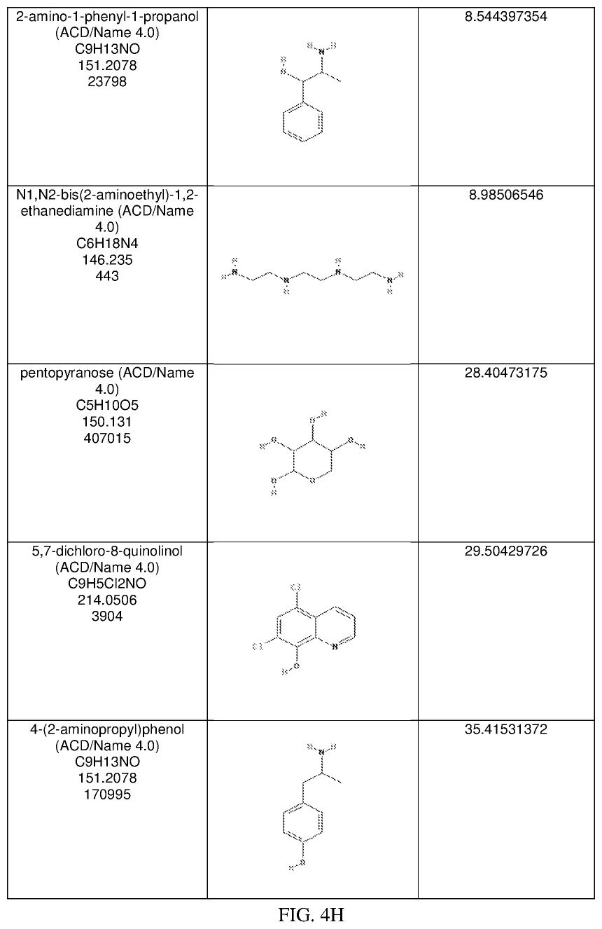

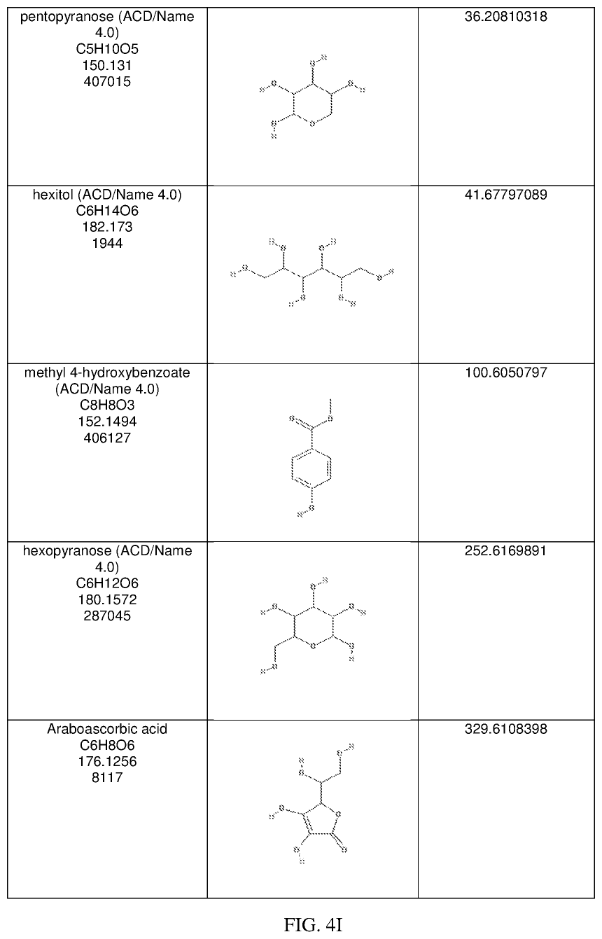

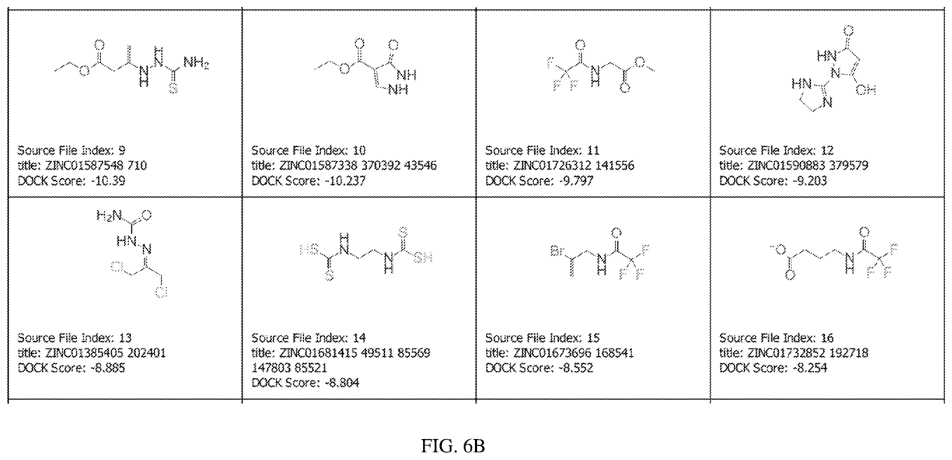

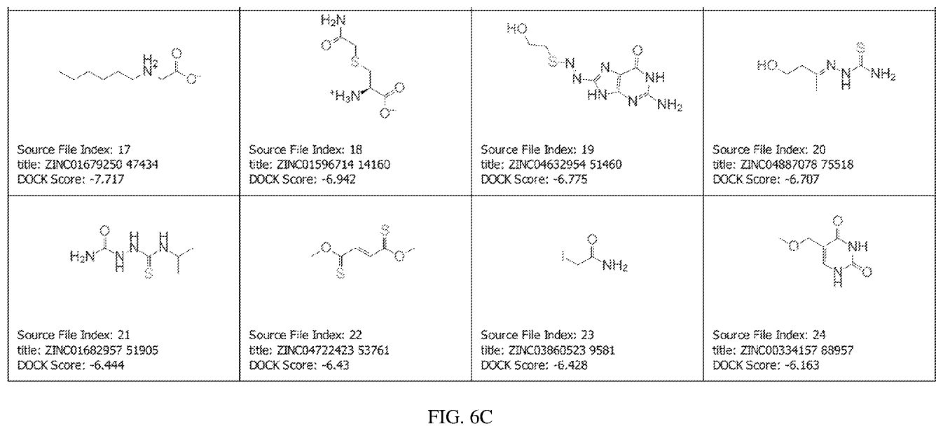

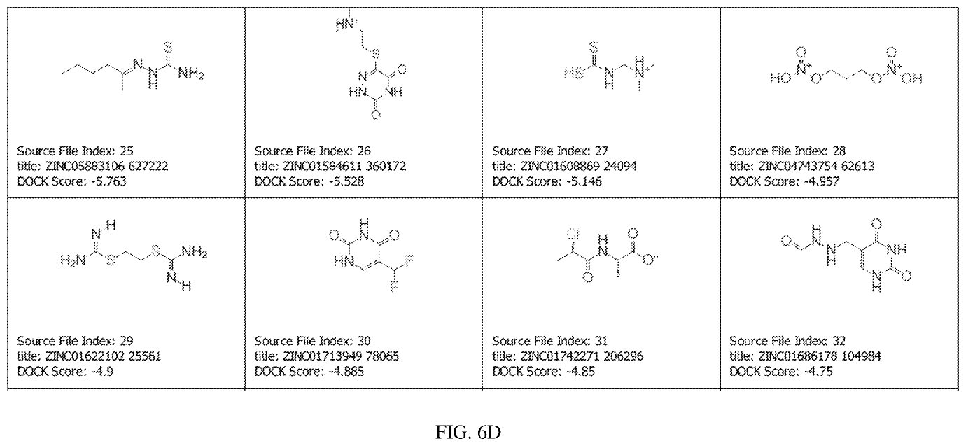

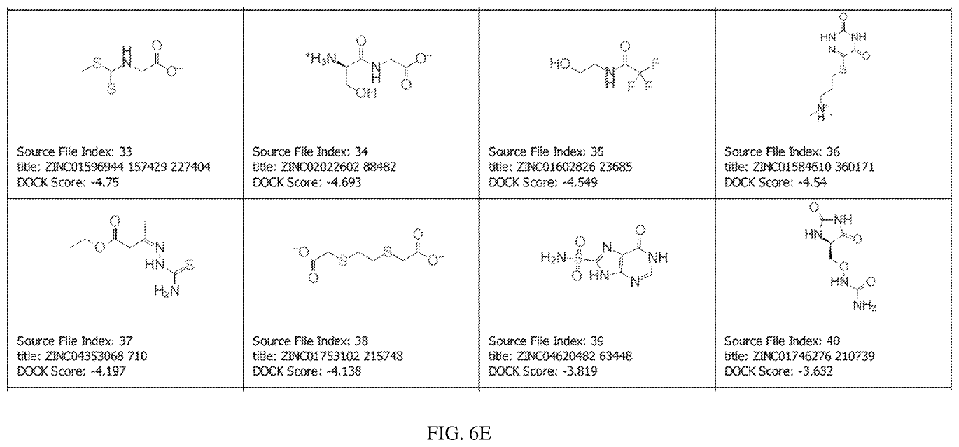

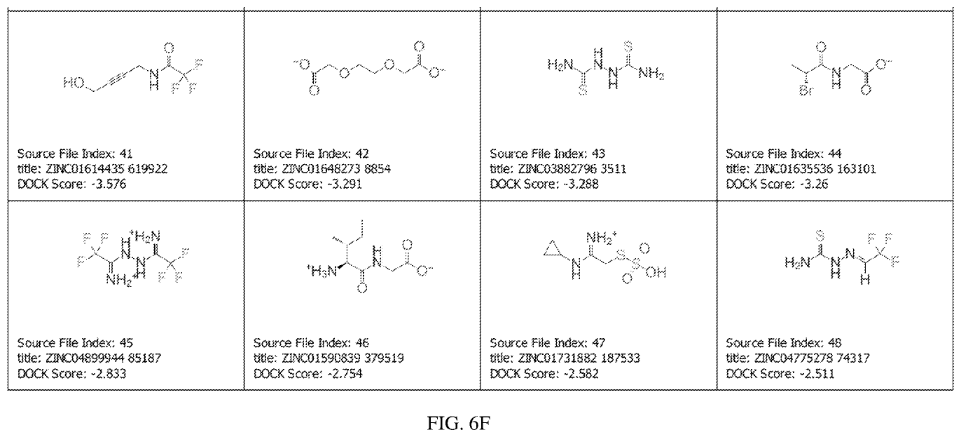

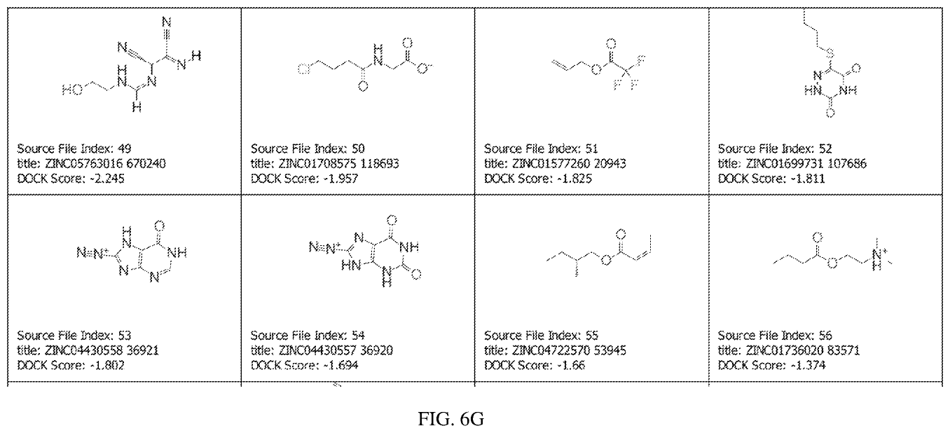

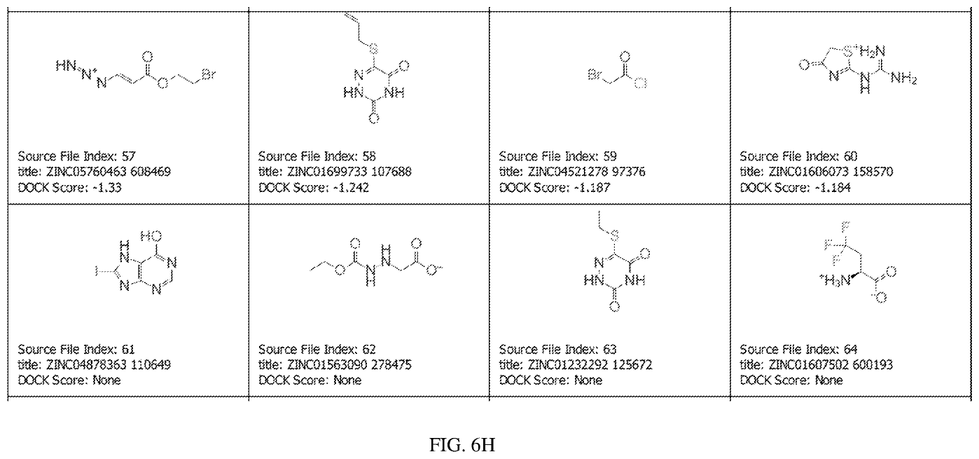

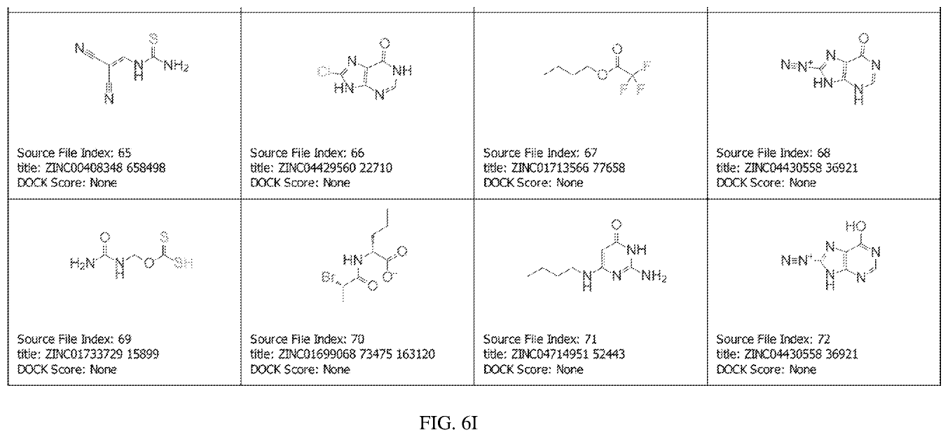

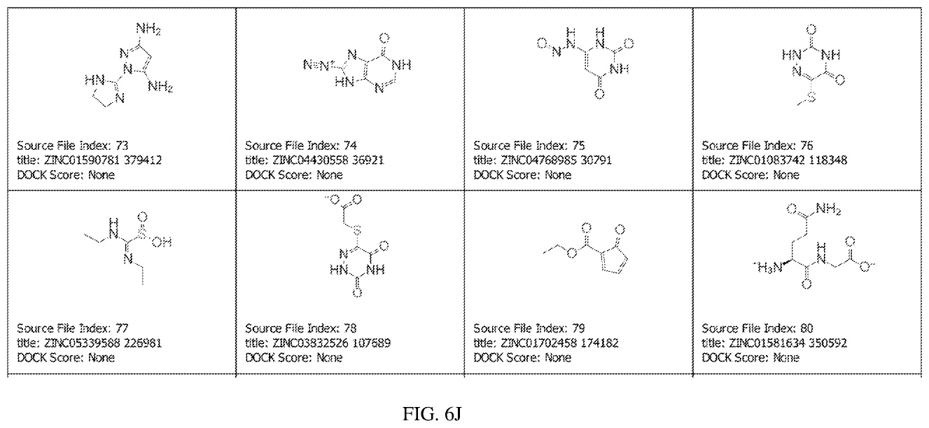

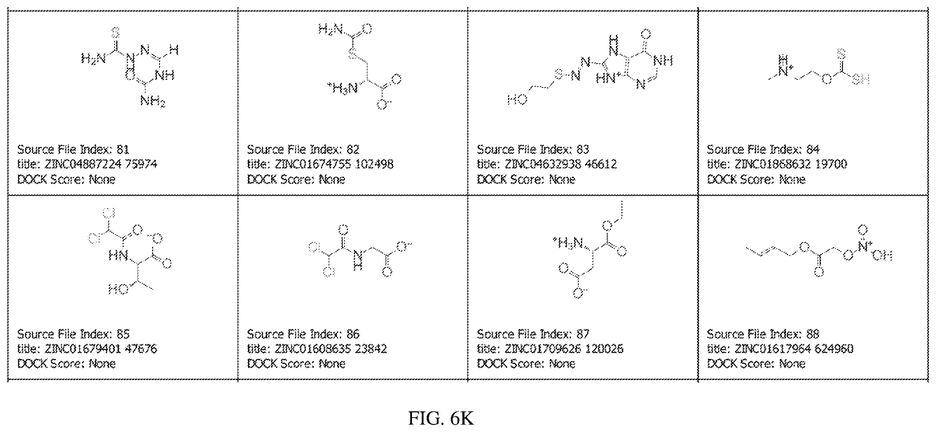

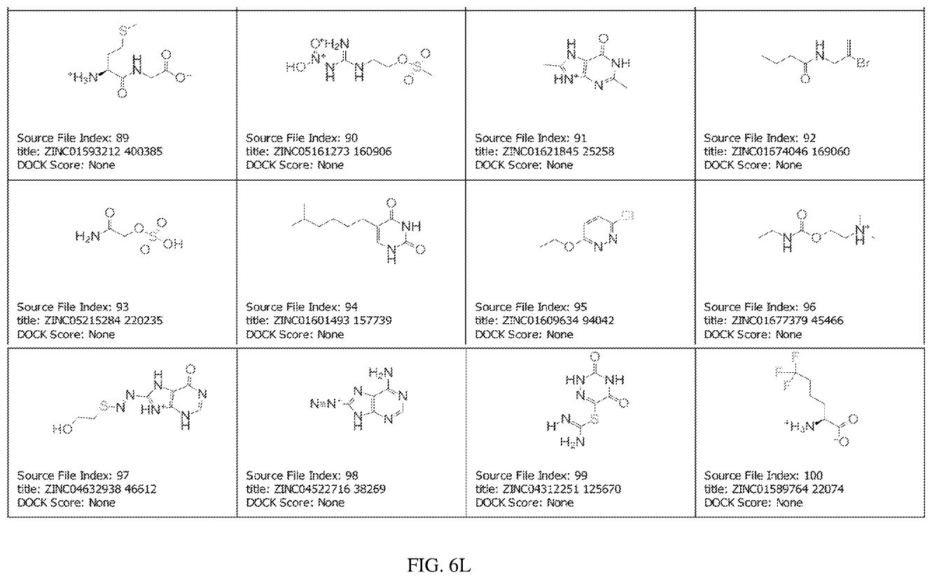

Non-limiting examples of a subject small molecule include an approved drug, for example an FDA-approved drug, a drug-like small molecule, a prodrug, or a metabolite of a drug. In some embodiments, the small molecule is a compound. In some embodiments, the compound is a drug. In some embodiments, the compound is an approved drug (e.g., FDA-approved or approved by other regulatory agency). In some embodiments, the compound is a drug not approved by a regulatory agency. In some embodiments, the compound is a drug-like small molecule. In some embodiments, the drug is a small molecule (e.g., a small molecule in Table 1, Table 2, FIGS. 4A-4I, or FIGS. 6A-6L). In some embodiments, the drug is any other compound capable of inducing an immune reaction in a subject, including but not limited to an immune hypersensitivity reaction. In some embodiments, the compound is one or more of those compounds found at e.g., https://zinc.docking.org/catalogs/home/. In some embodiments, a compound that interacts with an HLA molecule is administered to a subject.

There are multiple mechanisms by which drugs (e.g., small molecules) interact with HLA molecules. In some embodiments, the compound binds directly in the peptide binding groove (for example abacavir). In some embodiments, the compound does not bind directly in the peptide binding groove. In some embodiments, the compound influences the kinetics of peptide loading (for example by interacting with the HLA molecule). In some embodiments, the compound forms one or more covalent bonds with HLA molecule. In some embodiments, the compound binds the HLA molecule with Kd that is less than 50 μm (for example less than 49 μm, 48 μm, 47 μm, 46 μm, 45 μm, 44 μm, 43 μm, 42 μm, 41 μm, 40 μm, 39 μm, 38 μm, 37 μm, 36 μm, 35 μm, 34 μm, 33 μm, 32 μm, 31 μm, 30 μm, 29 μm, 28 μm, 27 μm, 26 μm, 25 μm, 24 μm, 23 μm, 22 μm, 21 μm, 20 μm, 19 μm, 18 μm, 17 μm, 16 μm, 15 μm, 14 μm, 13 μm, 12 μm, 11 μm, 10 μm, 9 μm, 8 μm, 7 μm, 6 μm, 5 μm, 4 μm, 3 μm, 2 μm, 1 μm, or less than 1 μm).

### Subjects

Some embodiments contemplated herein involve administering candidate compound to a subject. The term “subject,” “patient” and “individual” are used interchangeably herein and are intended to include living organisms in which an immune response (e.g., an immune hypersensitivity reaction) can be elicited (e.g., mammals). Examples of subjects include humans, dogs, cats, mice, rats, and transgenic species thereof. In some embodiments, a subject is a human subject. In some embodiments, a subject is non-human. In some embodiments, a subject is a mouse subject. In some embodiments, a subject is cells taken from a subject. In some embodiments, a subject is a subject having a disease (e.g., diagnosed as having a disease). In some embodiments, a subject is a subject having a cancer (e.g., diagnosed as having a cancer). In some embodiments, a subject is a subject having a higher than normal risk for developing cancer (e.g., identified as having a higher risk). In some embodiments, a subject is a subject that has been diagnosed as having a cancer. In some embodiments, a subject expresses one or more HLA alleles of interest (for example HLA-A2). In some embodiments, a subject expresses one or more HLA alleles associated with autoimmunity. In some embodiments, the subject exhibits immune hypersensitivity to a subject small molecule that preferentially or selectively binds to a specific HLA expressed by the subject.

In some embodiments, a compound useful to treat a cancer in a subject can prevent or slow the development, progression, and/or spread of a cancer in the subject. In some embodiments, a compound useful to treat a cancer in a subject can reduce the amount of cancer cells in a subject (e.g., by killing cancer cells in the subject).

### Use of Abacavir in the Treatment of Cancer

The HLA-B*57:01 genotype has a ˜0-20% incidence rate that is known to be different among different ethnicities. Abacavir (brand name Ziagen®) is contraindicated for patients having HLA-B*57:01. If taken by an HLA-B*57:01 patient, there is a significant (˜50%) risk of a hypersensitivity reaction, which can be quite severe and even fatal (Mallal, et al., N. Engl. J. Med. 2008; 358(6):568-79). The overall incidence rate of hypersensitivity to abacavir in the absence of genetic prescreening (i.e., including both HLA-B*57:01⁺ and HLA-B*57:01⁻ individuals) is ˜6% (Martin et al., Clin Pharmacol Ther. 2012 April; 91(4): 734-738).

Symptoms of a hypersensitivity reaction include at least two of the following: fever, rash, gastrointestinal symptoms (e.g., nausea, vomiting, abdominal pain), fatigue, cough, and/or dyspnea. Such symptoms increase in severity over time if administration of the drug (e.g., small molecule) is continued despite the progressive symptoms.

Since at least 2002, widespread screening for HLA-B*57:01 has been recommended by the FDA and other governing health bodies for all patients prior to starting abacavir therapy (Mallal et al., Lancet. 2002; 359(9308):727-32). If this HLA allelic variant is present in the HIV patient, the FDA recommends that an alternate drug (e.g., small molecule) be administered. If screening does not occur and a hypersensitivity reaction is elicited, immediate termination of abacavir therapy is recommended. Negative hypersensitivity symptoms increase in severity over time if administration of the drug is continued despite the progressive symptoms (Martin et al., Clin Pharmacol Ther. 2012 April; 91(4): 734-738).

Previous data have shown that peripheral blood mononuclear cells from hypersensitive HLA-B*57.01⁺ patients have a detectable immune response when cultured with abacavir in vitro (Almeida et al., Antivir Ther. 2008; 13(2):281-8). This immune response includes increased expression of interferon-γ, tumor necrosis factor-α, and other inflammatory cytokines.

The FDA-approved dose of orally administered abacavir for adults for treating symptoms associated with HIV is 600 mg/day.

The disclosure provides methods for systematically stimulating a strong immune response, or immune hypersensitivity reaction, to abacavir in HLA-B*57.01⁺ subjects (such as human subjects) that suffer from cancer. Contrary to FDA guidelines regarding the contraindication of HLA-B*57:01⁺ patients, the widespread use of pre-treatment genotyping to avoid administering abacavir to such patients, and recommendations to avoid or immediately halt abacavir treatment in HLA-B*57:01⁺ patients in view of hypersensitivity symptoms increasing in severity over time if administration is continued, the disclosure provides for continued abacavir treatment even after a hypersensitive reaction (along with possible additional “adverse” effects) is observed. In some embodiments, administration of abacavir is continued uninterrupted following observation of a stimulated immune response (e.g., an immune hypersensitivity reaction) and/or symptoms associated therewith. Additional doses may be administered that are below, or exceed, a level of discomfort for the subject. It is contemplated that hypersensitivity reactions in subjects will still be closely monitored by physicians, and the administrations terminated where appropriate.

In some embodiments, a first dose of abacavir of between 100 and 1,000 mg/day is administered. In some embodiments, a second dose of abacavir of between 100 and 1,000 mg/day is administered. In some embodiments, a third and/or additional doses of abacavir in this range are administered. In some embodiments, the first, second, third and additional doses are in the same amount. In some embodiments, the first, second, third and additional doses are in different amounts. In some embodiments, the first, second, third and/or additional are in the amount of about 100, 200, 300, 400, 500, 600, 700, 800, 900, or 1000 mg/day. In some embodiments, the second dose is reduced relative to the first dose. In some embodiments, the second dose is equal to the first dose. In some embodiments, the second dose is higher than the first dose. In some embodiments, the third dose is reduced relative to the first dose and/or second dose. In some embodiments, the third dose is higher than the first dose and/or the second dose. In some embodiments, dosing is continually raised until a severe hypersensitivity response is elicited.

In some embodiments, a first dose of abacavir of 600 mg/day is administered. In some embodiments, a second dose of abacavir of 600 mg/day is administered. In some embodiments, abacavir is administered in 20-day, 22-day, 24-day, 26-day, 28-day, 30-day, 40-day, 56-day, or 60-day cycles. In some embodiments, abacavir is administered in 28-day cycles. In some embodiments, the abacavir is administered orally. In some embodiments, the abacavir is administered sublingually, transdermally, intravenously, subcutaneously, intramuscularly, or by another route of administration known in the art.

In some embodiments, abacavir is administered in combination with one or more additional anti-cancer drugs and/or anti-cancer treatments. In some embodiments, abacavir is administered in combination with one or more epitopes (e.g., one or more cancer related peptide epitopes). In some embodiments, the one or more epitopes are patient-specific epitopes (e.g., personalized cancer vaccines). In some embodiments, abacavir is administered in combination with chemotherapy. In some embodiments, abacavir is administered in combination with biotherapy.

The desired hypersensitivity response of the disclosed methods of treatment comprising abacavir administrations may be evaluated by any method known in the art. In some embodiments, the response is evaluated by measuring T cell counts in the subject's peripheral blood or bone marrow. In some embodiments, the disclosed methods provide for elevation in T cell counts, WBC counts, absolute lymphocyte count, PBMC counts, B cell counts, macrophage counts, or dendritic cell counts.

In some embodiments, a patient treated with abacavir is a non-cancer patient who does not express the HLA-B*57:01 subtype. In some embodiments, the white blood cell count, absolute lymphocyte count, and/or absolute T cell count present in the blood (or a component of blood, e.g., serum) of said patient does not increase in response to the treatment with abacavir. In some embodiments, the white blood cell count of said patient is 4.5×10⁹/L to 11×10⁹/L following the treatment with abacavir. In some embodiments, the absolute lymphocyte count of said patient is 1×10⁹/L to 4×10⁹/L following the treatment with abacavir. In some embodiments, the absolute T cell count of said patient is 0.5×10⁹/L to 1.6×10⁹/L following the treatment with abacavir.

In some embodiments, the patient treated with abacavir is a non-cancer patient who expresses the HLA-B*57:01 subtype, but has no hypersensitivity reaction to said treatment. In some embodiments, the white blood cell count, absolute lymphocyte count, and/or absolute T cell count present in the blood (or a component of blood, e.g., serum) of said patient does not increase in response to the treatment with abacavir. In some embodiments, the white blood cell count of said patient is 4.5×10⁹/L to 11×10⁹/L following the treatment with abacavir. In some embodiments, the absolute lymphocyte count of said patient is 1×10⁹/L to 4×10⁹/L following the treatment with abacavir. In some embodiments, the absolute T cell count of said patient is 0.5×10⁹/L to 1.6×10⁹/L following the treatment with abacavir.

In some embodiments, the patient treated with abacavir is a non-cancer patient who expresses the HLA-B*57:01 subtype, and has a mild hypersensitivity reaction to said treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 6.75×10⁹/L to 16.5×10⁹/L following the treatment with abacavir. In some embodiments, the absolute lymphocyte count of said patient is 1.5×10⁹/L to 6×10⁹/L following the treatment with abacavir. In some embodiments, the absolute T cell count of said patient is 0.75×10⁹/L to 2.4×10⁹/L following the treatment with abacavir.

In some embodiments, the patient treated with abacavir is a non-cancer patient who expresses the HLA-B*57:01 subtype, and has a moderate hypersensitivity reaction to said treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 9×10⁹/L to 22×10⁹/L following the treatment with abacavir. In some embodiments, the absolute lymphocyte count of said patient is 2×10⁹/L to 8×10⁹/L following the treatment with abacavir. In some embodiments, the absolute T cell count of said patient is 1×10⁹/L to 3.2×10⁹/L following the treatment with abacavir.

In some embodiments, the patient treated with abacavir is a non-cancer patient who expresses the HLA-B*57:01 subtype, and has a severe hypersensitivity reaction to said treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 13.5×10⁹/L to 33×10⁹/L following the treatment with abacavir. In some embodiments, the absolute lymphocyte counx 10⁹/L t of said patient is 3×10⁹/L×10⁹/L to 12×10⁹/L×10⁹/L following the treatment with abacavir. In some embodiments, the absolute T cell count of said patient is 1.5×10⁹/L to 4.8×10⁹/L following the treatment with abacavir.

In some embodiments, the patient treated with abacavir is a cancer patient who does not express the HLA-B*57:01 subtype. In some embodiments, the white blood cell count, absolute lymphocyte count, and/or absolute T cell count present in the blood (or a component of blood, e.g., serum) of said patient does not increase in response to the treatment with abacavir. In some embodiments, the white blood cell count of said patient is 4.5×10⁹/L to 11×10⁹/L following the treatment with abacavir. In some embodiments, the absolute lymphocyte count of said patient is 1×10⁹/L to 4×10⁹/L following the treatment with abacavir. In some embodiments, the absolute T cell count of said patient is 0.5×10⁹/L to 1.6×10⁹/L following the treatment with abacavir.

In some embodiments, the patient treated with abacavir is a cancer patient who expresses the HLA-B*57:01 subtype and is not being treated with a second type of anti-cancer drug and/or treatment (e.g., chemotherapy or biotherapy), but has no hypersensitivity reaction to said abacavir treatment. In some embodiments, the white blood cell count, absolute lymphocyte count, and/or absolute T cell count present in the blood (or a component of blood, e.g., serum) of said patient does not increase in response to the treatment with abacavir. In some embodiments, the white blood cell count of said patient is 4.5×10⁹/L to 11×10⁹/L following the treatment with abacavir. In some embodiments, the absolute lymphocyte count of said patient is 1×10⁹/L to 4×10⁹/L following the treatment with abacavir. In some embodiments, the absolute T cell count of said patient is 0.5×10⁹/L to 1.6×10⁹/L following the treatment with abacavir.

In some embodiments, the patient treated with abacavir is a cancer patient who expresses the HLA-B*57:01 subtype and is not being treated with a second type of anti-cancer drug and/or treatment (e.g., chemotherapy or biotherapy), and has a mild hypersensitivity reaction to said abacavir treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 6.75×10⁹/L to 16.5×10⁹/L following the treatment with abacavir. In some embodiments, the absolute lymphocyte count of said patient is 1.5×10⁹/L to 6×10⁹/L following the treatment with abacavir. In some embodiments, the absolute T cell count of said patient is 0.75×10⁹/L to 2.4×10⁹/L following the treatment with abacavir.

In some embodiments, the patient treated with abacavir is a cancer patient who expresses the HLA-B*57:01 subtype and is not being treated with a second type of anti-cancer drug and/or treatment (e.g., chemotherapy or biotherapy), and has a moderate hypersensitivity reaction to said abacavir treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 9×10⁹/L to 22×10⁹/L following the treatment with abacavir. In some embodiments, the absolute lymphocyte count of said patient is 2×10⁹/L to 8×10⁹/L following the treatment with abacavir. In some embodiments, the absolute T cell count of said patient is 1 to 3.2×10⁹/L×10⁹/L following the treatment with abacavir.

In some embodiments, the patient treated with abacavir is a cancer patient who expresses the HLA-B*57:01 subtype and is not being treated with a second type of anti-cancer drug and/or treatment (e.g., chemotherapy or biotherapy), and has a severe hypersensitivity reaction to said abacavir treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 13.5×10⁹/L to 33×10⁹/L following the treatment with abacavir. In some embodiments, the absolute lymphocyte count of said patient is 3×10⁹/L to 12×10⁹/L following the treatment with abacavir. In some embodiments, the absolute T cell count of said patient is 1.5×10⁹/L to 4.8×10⁹/L following the treatment with abacavir.

In some embodiments, the patient treated with abacavir is a cancer patient who expresses the HLA-B*57:01 subtype and is being treated with at least a second type of anti-cancer drug and/or treatment (e.g., chemotherapy or biotherapy), but has no hypersensitivity reaction to said abacavir treatment. In some embodiments, the second type of anti-cancer drug and/or treatment lowers the white blood cell count, absolute lymphocyte count, and/or absolute T cell count present in the blood (or a component of blood, e.g., serum) of said patient, relative to a cancer patient who expresses the HLA-B*57:01 subtype and is not being administered a second type of anti-cancer drug and/or treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 2×10⁹/L to 8×10⁹/L following the treatment with abacavir. In some embodiments, the absolute lymphocyte count of said patient is 0.5×10⁹/L to 2×10⁹/L following the treatment with abacavir. In some embodiments, the absolute T cell count of said patient is 0.25×10⁹/L to 1×10⁹/L following the treatment with abacavir.

In some embodiments, the patient treated with abacavir is a cancer patient who expresses the HLA-B*57:01 subtype and is being treated with at least a second type of anti-cancer drug and/or treatment (e.g., chemotherapy or biotherapy), and has a mild hypersensitivity reaction to said abacavir treatment. In some embodiments, the second type of anti-cancer drug and/or treatment lowers the white blood cell count, absolute lymphocyte count, and/or absolute T cell count present in the blood (or a component of blood, e.g., serum) of said patient, relative to a cancer patient who expresses the HLA-B*57:01 subtype and is not being administered a second type of anti-cancer drug and/or treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 4×10⁹/L to 11×10⁹/L following the treatment with abacavir. In some embodiments, the absolute lymphocyte count of said patient is 1×10⁹/L to 4×10⁹/L following the treatment with abacavir. In some embodiments, the absolute T cell count of said patient is 0.5×10⁹/L to 2×10⁹/L following the treatment with abacavir.

In some embodiments, the patient treated with abacavir is a cancer patient who expresses the HLA-B*57:01 subtype and is being treated with at least a second type of anti-cancer drug and/or treatment (e.g., chemotherapy or biotherapy), and has a moderate hypersensitivity reaction to said abacavir treatment. In some embodiments, the second type of anti-cancer drug and/or treatment lowers the white blood cell count, absolute lymphocyte count, and/or absolute T cell count present in the blood (or a component of blood, e.g., serum) of said patient, relative to a cancer patient who expresses the HLA-B*57:01 subtype and is not being administered a second type of anti-cancer drug and/or treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 6×10⁹/L to 24×10⁹/L following the treatment with abacavir. In some embodiments, the absolute lymphocyte count of said patient is 1.5×10⁹/L to 6×10⁹/L following the treatment with abacavir. In some embodiments, the absolute T cell count of said patient is 0.75×10⁹/L to 3×10⁹/L following the treatment with abacavir.

In some embodiments, the patient treated with abacavir is a cancer patient who expresses the HLA-B*57:01 subtype and is being treated with at least a second type of anti-cancer drug and/or treatment (e.g., chemotherapy or biotherapy), and has a severe hypersensitivity reaction to said abacavir treatment. In some embodiments, the second type of anti-cancer drug and/or treatment lowers the white blood cell count, absolute lymphocyte count, and/or absolute T cell count present in the blood (or a component of blood, e.g., serum) of said patient, relative to a cancer patient who expresses the HLA-B*57:01 subtype and is not being administered a second type of anti-cancer drug and/or treatment. In some embodiments, the white blood cell count, absolute lymphocyte count, and/or absolute T cell count present in the blood (or a component of blood, e.g., serum) of said patient increases in response to the treatment with abacavir. In some embodiments, the white blood cell count of said patient is 8×10⁹/L to 24×10⁹/L following the treatment with abacavir. In some embodiments, the absolute lymphocyte count of said patient is 2×10⁹/L to 8×10⁹/L following the treatment with abacavir. In some embodiments, the absolute T cell count of said patient is 1×10⁹/L to 4×10⁹/L following the treatment with abacavir.

The disclosed methods may provide for treatment or amelioration of symptoms of a cancer in the subject. These methods may provide for reduction in the size of a tumor in the subject, or reduction in percentage of cancer cells within a tissue, such as reduction of malignant blast cells in bone marrow or shrinkage of malignant lymph nodes. These methods may prolong survival times or improve symptoms in subjects suffering from cancer. These methods may also assist in preventing the onset or mitigating the severity of cancer symptoms. Additional methods known in the art for assessing the degree of treatment or amelioration of cancer include measuring the overall survival (OS) rate, OS time, Progression Free Survival (PFS) time, PFS rate, and Measurable Residual Disease (MRD) by a number of different assays including but not limited to flow cytometry, cytogenetics, or next generation sequencing. These measurements may be administered at 1, 2, 3, 6, 9, 12, 15, 18, or 24 months. In some embodiments, these measurements are administered at 3, 6, 9 and 12 months. Additional methods for assessing the therapeutic response of the disclosed methods comprise morphologic remission rate at various time points, time to achieve morphologic remission, cytogenetic remission rate at various time points, time to achieve cytogenetic remission, molecular (genetic) remission rate at various time points, time to achieve molecular remission, progression free survival rate at various time points, and progression free survival time. Such various time points may be 1, 2, 3, 6, 9, 12, 15, 18, or 24 months, optionally 3, 6, 9 and 12 months.

Not wishing to be bound by a particular theory, higher cell surface expression of HLA-B*57:01 protein may correlate with greater cancer disease regression. Accordingly, in some embodiments, provided herein are methods of determining the effectiveness of an abacavir treatment comprising administering a first and/or additional doses of abacavir, and further a step of measuring an association of abacavir efficacy with a degree of HLA-B*57:01 cell surface expression on malignant blasts, or immature white blood cells. In some embodiments, these methods further comprise a step of determining one or more cancer microenvironment molecular “signatures” in the bone marrow or peripheral blood (PB) of a subject, and thus comprise the step of taking a bone marrow aspirate, biopsy, and/or a peripheral blood sample from the subject following abacavir administration or administration of other drug-allele pair that is intended to elicit a hypersensitivity reaction. In some embodiments, the subject's pool of lymphocytes, quantified by various methods including WBC count, absolute lymphocyte count, T cell count, B cell count, macrophage count, dendritic cell count, may serve to identify subjects who are candidates to receive abacavir administration or administration of other drug-allele pair that is intended to elicit a hypersensitivity reaction.

### Method of Identifying Candidate Compounds Based on Predicted HLA Binding Affinity

Methods contemplated herein involve identifying, from a number of candidate compounds (e.g., 100,000 candidate compounds), those compounds which are most likely, based on the methods disclosed herein, to bind to, or otherwise stimulate an immune response from, HLA molecules. In some embodiments, the HLA molecules of interest are selected based on known association with a disease (e.g., certain mutagenic forms of cancer, Graft-Versus-Host disease, etc.). In some embodiments, the HLA molecules of interest are selected based on certain structural features (e.g., binding pockets). In some embodiments, the HLA molecules of interest contain structural features of interest that are known to be conserved among one of more HLA alleles (see, e.g., https://bmcimmunol.biomedcentral.com/articles/10.1186/1471-2172-9-1). In some embodiments, the HLA molecule is an HLA-B molecule. In some embodiments, the HLA-B molecule has Y at position 9 and L at position 156.

In some embodiments, the methods contemplated herein identify those candidate compounds most likely to bind to, or otherwise stimulate an immune response from, HLA molecules based on a set of criteria. In some embodiments, the candidate compounds are identified based upon their predicted binding affinity to the HLA molecule(s) of interest. In some embodiments, the predicted binding affinity is calculated based on in silico modeling of the structural features of the HLA molecule(s) of interest. In some embodiments, the in silico modeling identifies active compounds (e.g., those compounds predicted to bind the HLA molecule of interest that actually do bind the HLA molecule of interest) with 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% accuracy. In some embodiments, the in silico modeling identifies active compounds with 5-10% accuracy. Thus, in some embodiments wherein forty (40) candidate compounds are selected based upon the in silico modeling as described herein, 2-4 candidate compounds successfully bind the HLA molecule of interest in in vitro, ex vivo, or in vivo testing as further described herein.

In some embodiments, the predicted binding affinity is calculated using scoring grid DOCK (http://dock.compbio.ucsf.edu/) (UCSF) to generation a score that represents the predicted binding affinity for each candidate compound to the HLA molecule of interest. In some embodiments, the calculated score is a delta G (ΔG) value. In some embodiments, top scoring compounds are selected for further analysis (e.g., compounds with the most negative ΔG values). In some embodiments, compounds with ΔG values around −40 kcal per mol (for example ranging from around −20 to around −80 kcal per mol) are selected. In some embodiments, compounds with ΔG values below −40 kcal per mol are selected. In some embodiments, a more negative ΔG value indicates a higher predicted binding affinity. In some embodiments, candidates with the forty or so most negative ΔG values are selected for further testing. In some embodiments, the candidates with the most negative ΔG values have ΔG values of −40 kcal per mol or more negative values.

In some embodiments, other criteria are used to identify those candidate compounds most likely to bind to, or otherwise stimulate an immune response from, HLA molecules, for example the known association of a particular compound with an HLA allele of interest.

Thus, in some embodiments, a method for identifying candidate compounds based on predicted HLA binding affinity is contemplated, comprising one or more of the following steps:
(1) in silico modeling of the structure of the HLA molecule of interest using the methods described herein;
(2) structural superposition of the compound and HLA molecule of interest using LSQKAB in CCP4;
(3) selection of a potential binding site on the HLA molecule of interest using SPHGEN in DOCK;
(4) calculation of a scoring grid DOCK;
(5) parallel processing to predict molecular docking DOCK;
(6) obtaining compounds of interest (and, e.g., dissolving them in a solvent such as dimethyl sulfoxide);
(7) testing the top scoring compounds in vitro or ex vivo using biochemical and cellular assays as described herein; and/or
(8) testing the top scoring compounds in vivo.

### Use of Identified Candidate Compounds in the Treatment of Disease

In some embodiments, one or more of the compounds listed in Table 1, Table 2, FIGS. 4A-4I, or FIGS. 6A-6L can be administered to treat a subject having a disease. In some embodiments, the one or more compounds are useful for treating subjects expressing the HLA allele(s) that correspond to (e.g., are activated by) the compound. In some embodiments, the one or more compounds stimulate a T cell response in subjects expressing the HLA alleles that correspond to the compound.

In some embodiments, a compound is administered in an amount effective to treat a disease in the subject (for example a cancer in the subject). In some embodiments, a compound is administered alone. In some embodiments, a compound is administered in combination with one or more additional anti-cancer drugs. In some embodiments, a compound is administered in combination with one or more epitopes (e.g., one or more cancer related peptide epitopes). In some embodiments, the one or more epitopes are patient-specific epitopes (e.g., personalized cancer vaccines).

In some embodiments, a compound that interacts with an HLA allele that is expressed in a subject (e.g., in a subject diagnosed as having cancer) is administered to the subject (e.g., to treat cancer in the subject). In some embodiments, the HLA allele is one of an HLA allele supertype (for example a groupings of HLA alleles based upon a common structural characteristic e.g., a particular binding pocket). In some embodiments, a compound that interacts with an HLA allele supertype that is expressed in a subject (e.g., in a subject diagnosed as having cancer) is administered to the subject (e.g., to treat cancer in the subject). Compounds that interact with several HLA alleles within an HLA allele supertype can be used to treat subjects expressing different HLA alleles if the alleles are within the HLA allele supertype. In contrast, compounds that interact with only one HLA allele are useful to treat subjects that express that particular allele.

In some embodiments, two or more different compounds are administered to a subject, wherein each compound specifically binds to a different HLA allele expressed in the subject.

In some embodiments, the subject is a cancer patient with Graft-Versus-Host disease. In some embodiments, the subject has excessive CD8+ T cell activity. In some embodiments, two or more different compounds that bind HLA-A, HLA-B, and HLA-C molecules of the subject are administered to the subject. In some embodiments, the two or more different compounds administered to the subject block peptide binding (for example in HLA-DQ8). In some embodiments, the two or more different compounds administered to the subject block all class I HLA molecules expressed in the subject.

In other embodiments, the two or more different compounds administered to the subject boost immunity to tumors. In some embodiments, the two or more different compounds administered to the subject facilitate peptide binding (for example abacavir). In some embodiments, a subject expressing HLA-B*57 and HLA-B*58 (e.g., HLA-B*57:01 and HLA-B*58:01) is administered a combination of abacavir and allopurinol. In some embodiments, the two or more different compounds administered to the subject are beneficial to the treatment of disease (e.g., the symptoms of the disease are alleviated as a result of the administration).

In some embodiments, “administering” or “administration” means providing a material to a subject in a manner that is pharmacologically useful. In some embodiments, one or more compounds are administered to a subject enterally. In some embodiments, an enteral administration of one or more compounds are oral. In some embodiments, one or more compounds are administered to the subject parenterally. In some embodiments, one or more compounds are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, one or more compounds are administered to the subject by injection into the hepatic artery or portal vein.

To “treat” a disease as the term is used herein, means to reduce the frequency or severity of at least one sign or symptom of a disease or disorder experienced by a subject. The compounds and compositions described above or elsewhere herein are typically administered to a subject in an effective amount, that is, an amount capable of producing a desirable result. The desirable result will depend upon the active agent being administered. For example, an effective amount of a compound may be an amount of the compound that is capable of inducing a response in a host organ, tissue, or cell. A therapeutically acceptable amount may be an amount that is capable of treating a disease, e.g., a cancer. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, the disease is a cancer. The term “cancer” as used herein is defined as disease characterized by the rapid and uncontrolled growth of aberrant cells. In an embodiment, an uncontrolled growth of aberrant cells can be benign. Cancer cells of a subject cancer can spread locally or through the bloodstream and lymphatic system to other parts of the body. In an embodiment, a subject cancer is non-metastatic. In an embodiment, a subject cancer is metastatic. Subject cancers can be present in adult subject or pediatric subjects. In an embodiment, a cancer is a pediatric cancer. In an embodiment, a cancer is present in an adult subject.

Subject cancers can be solid cancers or liquid cancer. In some embodiments, the cancer is skin cancer, bladder cancer, bone cancer, brain cancer, central nervous system (CNS) cancer, gastro-intestinal cancer, breast cancer, cervical cancer, colon cancer, rectum cancer, connective tissue cancer, esophageal cancer, eye cancer, kidney cancer, larynx cancer, liver cancer, lung cancer, Hodgkin's lymphoma, non-Hodgkin's lymphoma, basal cell carcinoma, melanoma, myeloma, multiple myeloma, mesothelioma, leukemia, oral cavity cancer, ovarian cancer, pancreatic cancer, prostate cancer, rhabdomyosarcoma, skin cancer, stomach cancer, testicular cancer, endometrial cancer, neoplasia, and/or uterine cancer. In some embodiments, the cancer is a lung cancer. In some embodiments, the cancer is a melanoma. In an embodiment, a subject with melanoma comprises a V600E mutation.

In some cases, a subject cancer comprises a tumor-associated antigen. Tumor-associated antigens can be antigens not normally expressed by the subject; they can be mutated, truncated, misfolded, or otherwise abnormal manifestations of molecules normally expressed by the subject. In some cases, tumor-associated antigens can be identical to molecules normally expressed but expressed at abnormally high levels; or they can be expressed in a context or environment that is abnormal. Tumor-associated antigens can be proteins or functional fragments thereof, complex carbohydrates, gangliosides, haptens, nucleic acids, other biological molecules, or any combinations thereof. In another embodiment, a subject cancer is a mutagenic cancer. Exemplary mutagenic cancers can be associated with neo-antigens that arise as a result of mutations, such as somatic mutations.

In some embodiments, the cancer is a mutagenic cancer associated with a neo-antigen. A neo-antigen can arise from a gene or portion thereof that can comprise a mutation that gives rise to a neoantigen or neoepitope.

In an embodiment, a cancer cell is from a tumor stroma from a tumor microenvironment. Tumor stroma can contain cancer cells that express stomal antigens. Exemplary tumor stromal antigens can be present on, for example, tumor endothelial cells, tumor vasculature, tumor fibroblasts, tumor pericytes, tumor stroma, and/or tumor mesenchymal cells.

In some embodiments, the disease is a virus. In some embodiments, the candidate compounds identified using the method described herein enhance virus recognition when administered to a subject. Thus, in some embodiments, the candidate compounds identified using the method described herein improve innate immunity to disease (e.g., viruses).

Some embodiments contemplate the use of the candidate compounds identified using the method described herein in a composition comprising the composition in combination with other therapies for the purpose of treating a subject with a disease in need of such treatment. In some embodiments, the composition comprises a pharmaceutically acceptable carrier.

Throughout the present disclosure, references to “a compound” and “an HLA binding molecule” provided herein are intended to encompass the compound or group of compounds, and also pharmaceutically acceptable salts, stereoisomers, tautomers, isotopically labeled derivatives, solvates, hydrates, polymorphs, co-crystals, and prodrugs thereof as described herein.

The term “carrier” refers to a diluent, adjuvant, excipient, or vehicle with which the one or more compounds and other therapies are administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for the use of one or more compounds and other therapies in human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

Typically, such compositions may contain at least about 0.1% of the therapeutic agent (e.g., the one or more compounds and/or other therapies) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., the one or more compounds and/or other therapies) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. In some embodiments, the form is sterile. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subretinal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

In some embodiments, the candidate compounds are administered in a composition comprising the composition in combination with other therapies to a subject. In some embodiments, the other therapy is an antibody that blocks the programmed death 1 receptor (PD-1) and/or its ligands (e.g., PD-L1). For non-limiting examples of such antibodies, see e.g. EP App. No. 10705120.3. The PD-1 cytoplasmic domain contains two tyrosines, one that constitutes an immunoreceptor tyrosine inhibitory receptor (ITIM) and the other one an immunoreceptor tyrosine-based switch motif (ITSM). The phosphorylation of the second tyrosine leads to the recruitment of the tyrosine phosphatases SHP2 and to some extent SHP1. These phosphatases will dephosphorylate ZAP70, CD3ζ and PKC θ and consequently will attenuate T cell signals. PD-1 mainly inhibits T and B cell proliferation by causing cell arrest in G0/G1 and inhibiting cytokine production in T cells. Two PD-1 ligands have been described, PD-L1/B7H1/CD274 and PD-L2/B7-DC/CD273. PD-L1 is expressed at low levels on immune cells such as B cells, dendritic cells, macrophages and T cells and is up regulated following activation. PD-L1 is also expressed on non-lymphoid organs such as endothelial cells, heart, lung, pancreas, muscle, keratinocytes and placenta. The expression within non lymphoid tissues suggests that PD-L1 may regulate the function of self-reactive T and B cells as well as myeloid cells in peripheral tissues or may regulate inflammatory responses in the target organs.

In some embodiments, the other therapy is chemotherapy. As used herein, chemotherapy refers to the administration of one or more compounds known to treat cancer to a subject in need of such treatment. Chemotherapy can be adjuvant or neoadjuvant chemotherapy, and includes the administration of any chemotherapeutic drug that has been shown effective for the treatment of the particular cancer. Thus, chemotherapeutic drugs include anthracycline derivatives, such as doxorubicin or adriamycin; taxane derivatives, such as paclitaxel or docetaxel; topoisomerase inhibitors, such as camptothecin, topotecan, irinotecan, 20-S-camptothecin, 9-nitro-camptothecin, 9-amino-camptothecin, or GI147211; and inhibitors of nucleotide biosynthesis, such as methotrexate and/or 5-fluorouracil (5-FU). In another embodiment, a chemotherapy can comprise anti-neoplastic agents such as alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other anti-neoplastic agents can be antibiotics, e.g., doxorubicin, bleomycin, dactinomycin, daunorubicin, mithramycin, mitomycin, mytomycin C, and daunomycin. Still other anti-neoplastic agents can be mitotic inhibitors (e.g., vinca alkaloids). These include vincristine, vinblastine and etoposide. Miscellaneous anti-neoplastic agents include taxol and its derivatives, L-asparaginase, dacarbazine, azacytidine, amsacrine, melphalan, VM-26, ifosfamide, mitoxantrone, and vindesine.

In some embodiments, the other therapy is an anti-cancer vaccine. In some embodiments, an anti-cancer vaccine is a composition comprising an antigen in association with an effective amount of at least one immunomodulator chemotherapeutic adjuvant eliciting an immune response in a patient and a pharmaceutically acceptable carrier. For non-limiting examples of anti-cancer vaccines, see e.g. US Patent App. No. 2010/0272676 A1. In some embodiments, the anti-cancer vaccine is capable of eliciting an immunoprotective response against a cancer. In some embodiments, the cancer is skin cancer, bladder cancer, bone cancer, brain cancer, CNS cancer, gastro-intestinal cancer, breast cancer, cervical cancer, colon cancer, rectum cancer, connective tissue cancer, esophageal cancer, eye cancer, kidney cancer, larynx cancer, liver cancer, lung cancer, Hodgkin's lymphoma, non-Hodgkin's lymphoma, basal cell carcinoma, melanoma, myeloma, multiple myeloma, mesothelioma, leukemia, oral cavity cancer, ovarian cancer, pancreatic cancer, prostate cancer, rhabdomyosarcoma, skin cancer, stomach cancer, testicular cancer, endometrial cancer, neoplasia, and/or uterine cancer.

In some embodiments, the other therapy is an antibody or an antibody fragment. The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

In some embodiments, the other therapy is a monoclonal antibody. Monoclonal antibodies are known in the art, and are homogenous preparations of antibodies (or fragments of antibodies) in which every antibody in the product is identical in its protein sequence, and thus every antibody is expected to have the same antigen recognition site, affinity, biologic interactions, and downstream biologic effects. For a non-limiting review on monoclonal antibodies, see e.g., Rajewsky, The advent and rise of monoclonal antibodies, Nature (2019). In some embodiments of the invention, the monoclonal antibody has been approved by the FDA for use as a therapeutic. In other aspects of the invention, the monoclonal antibody is one that is undergoing testing for use as a therapeutic or has potential for use as a therapeutic. A number of monoclonal antibodies have been approved by the FDA for therapeutic use including, but not limited to abciximab, adalimumab, adotrastuzumab emtansine, alemtuzumab, alirocumab, atezolizumab, avelumab, basiliximab, belimumab, bevacizumab, bezlotoxumab, blinatumomab, brentuximab vedotin, broadalumab, canakinumab, capromab pendetide, certolizumab pegol, cetuximab, daclizumab, daratumumab, densosumab, dinutuximab, durvalumab, elotuzumab, evolocumab, golimumab, infliximab, ipilimumab, ixekizumab, mepolizumab, natalizumab, necitumumab, nivolumab, obinutuzumab, ocrelizumab, ofatumumab, olaratumab, pertuzumab, ramucirumab, rituximab, siltuximab, tocilizumab, trastuzumab, ustekinumab, vedolizumab, sarilumab, and benralizumab. FDA-approved therapeutic mAbs contain variable regions that are mouse, rat, or human.

In some embodiments, the other therapy is a bispecific antibody. A “bispecific antibody,” as used herein, refers to an antibody having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368.

In some embodiments, the other therapy comprises an anti-angiogenic agent. Suitable anti-angiogenic agents for use in the disclosed methods and compositions include anti-VEGF antibodies, including humanized and chimeric antibodies, anti-VEGF aptamers, and antisense oligonucleotides. Other inhibitors of angiogenesis include angiostatin, endostatin, interferons, interleukin 1 (including α and β), interleukin 12, retinoic acid, and tissue inhibitors of metalloproteinase-1 and -2 (TIMP-1 and -2). Small molecules, including topoisomerases such as razoxane, a topoisomerase II inhibitor with anti-angiogenic activity, can also be used.

In some embodiments, the other therapy is one or more T cells that have been modified to express a chimeric antigen receptor (CAR). CARs are molecules that combine antibody-based specificity for a desired antigen (e.g., tumor antigen) with a T cell receptor-activating intracellular domain to generate a chimeric protein that exhibits a specific anti-tumor cellular immune activity. In an embodiment, a CAR comprises at least one extracellular targeting domain, at least one transmembrane domain, and at least one intracellular signaling domain. In some cases, a CAR comprises a hinge domain. A CAR extracellular targeting domain can be, comprise, or be derived from, for example, a monoclonal antibody, a recombinant antibody, a human antibody, a humanized antibody, or a functional derivative, variant, or fragment thereof, including, but not limited to, a heavy chain variable domain (VH), a light chain variable domain (VL), a Fab, a Fab′, a F(ab′)2, an Fv, a single-chain Fv (scFv), a minibody, a diabody, a single-domain antibody such as a VHH, or any combination thereof.

In some embodiments, a composition comprising one or more of the compounds shown in Table 1, FIGS. 4A-4I, or FIGS. 6A-6L is administered to a subject (e.g., a subject that has a disease).

In some embodiments, a composition comprising abacavir is administered to a subject (e.g., a subject that has a disease) that expresses HLA-B*57:01.

In some embodiments, a composition comprising one or more of the compounds shown in FIGS. 6A-6L is administered to a subject (e.g., a subject that has a disease) that expresses HLA-DR3.

### Assays

In some embodiments, one or more candidate compounds selected using the methods described herein are tested in vitro, ex vivo, and/or in vivo. In some embodiments, one or more candidate compounds selected using the methods described herein are tested using one or more assays. In some embodiments, the assay comprises a cell-based assay.

In some embodiments, the assay comprises an immuno-based assay. In some embodiments, the immuno-based assay comprises an isolated antibody specific for an antigen. In some embodiments, the assay comprises a nucleic acid-based assay, such as in-situ hybridization (e.g., FISH) or RT-PCR (e.g., quantitative RT-PCR or strand specific quantitative RT-PCR). Assays known in the art for detecting proteins and RNAs (see, e.g., Molecular Cloning: A Laboratory Manual, J. Sambrook, et al., eds., Third Edition, Cold Spring Harbor Laboratory Press, Cold Spring Harbor, N.Y., 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York) can be used alone or in combination with other methods of detection. Assays for detecting protein levels include, but are not limited to, immunoassays (also referred to herein as immune-based or immuno-based assays, e.g., Western blot, immunohistochemistry and ELISA assays), Mass spectrometry, and multiplex bead-based assays. Such assays for protein level detection are well-known in the art.

TABLE 1
Exemplary compounds.

| Drug (e.g., small molecule) | Structures |
|---|---|
| Abacavir | 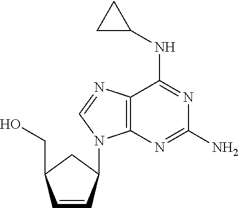 [SMILES: Nc1nc(NC2CC2)c2ncn([C@H]3C=C[C@@H](CO)C3)c2n1] |
| Allopurinol/Oxypurinol | 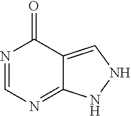 |
| | 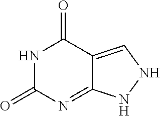 [SMILES: O=c1nc2[nH][nH]cc-2c(=O)[nH]1] |
| Carbamazepine | 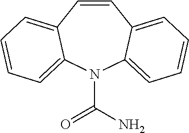 [SMILES: NC(=O)N1c2ccccc2C=Cc2ccccc21] |
| Feprazone | 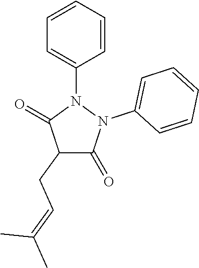 [SMILES: CC(C)=CCC1C(=O)N(c2ccccc2)N(c2ccccc2)C1=O] |
| Flucioxacillin | 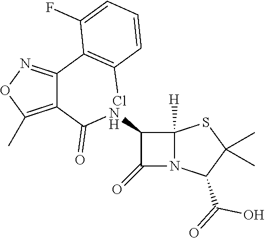 [SMILES: Cc1onc(-c2c(F)cccc2Cl)c1C(=O)N[C@@H]1C(=O)N2[C@@H]1SC(C)(C)[C@@H]2C(=O)O] |
| Sulfamethoxazole | 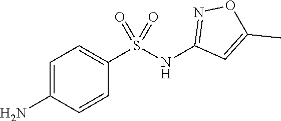 [SMILES: Cc1cc(NS(=O)(=O)c2ccc(N)cc2)no1] |
| Sulfonamides (drug class) | Varying structures |
| | 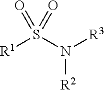 |
| Levamisole | 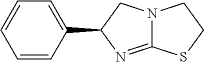 [SMILES: c1ccc([C@H]2CN3CCSC3=N2)cc1] |
| Oxicam | 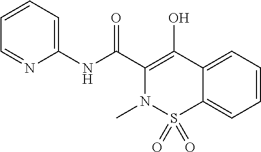 [SMILES: CN1C(C(=O)Nc2ccccn2)=C(O)c2ccccc2S1(=O)=O] |
| Phenytoin | 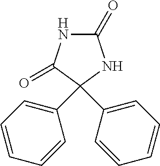 [SMILES: O=C1NC(=O)C(c2ccccc2)(c2ccccc2)N1] |
| Aspirin | 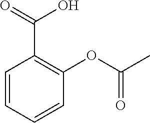 |
| Hydralazine | 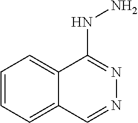 [SMILES: NNc1nncc2ccccc12] |
| Lapatinib | 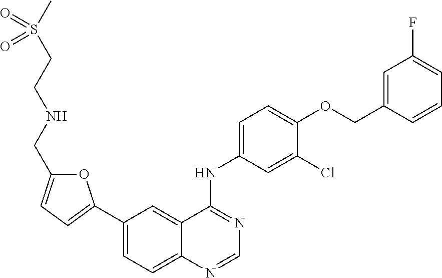 [SMILES: CS(=O)(=O)CCNCc1ccc(-c2ccc3ncnc(Nc4ccc(OCc5cccc(F)c5)c(Cl)c4)c3c2)o1] |
| NSAIDs (drug class) | Varying structures |
| Ximelagatran | 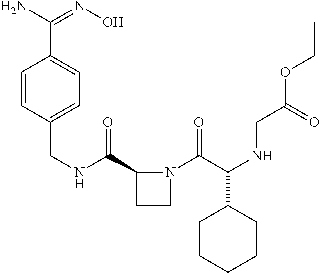 [SMILES: CCOC(=O)CN[C@@H](C(=O)N1CC[C@H]1C(=O)NCc1ccc(/C(N)=N\O)cc1)C1CCCCC1] |
| Aminopenicillins (drug class) | 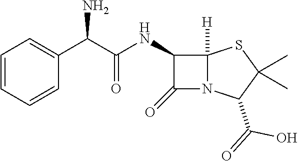 [SMILES: CC1(C)S[C@@H]2[C@H](NC(=O)[C@H](N)c3ccccc3)C(=O)N2[C@H]1C(=O)O] |
| Clozapine | 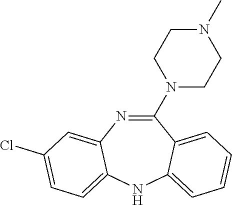 [SMILES: CN1CCN(C2=Nc3cc(Cl)ccc3Nc3ccccc32)CC1] |
| d-penicillamine | 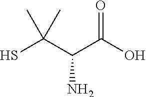 |
| Au+ Na+ Thiomalate | 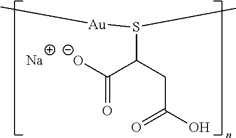 |
| Nevirapine | 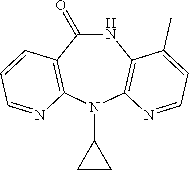 [SMILES: Cc1ccnc2c1NC(=O)c1cccnc1N2C1CC1] |
| Methazolamide | 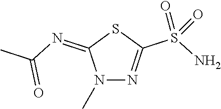 |
| Ticlopine / Ticlopidine | 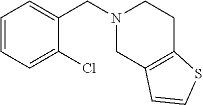 [SMILES: Clc1ccccc1CN1CCc2sccc2C1] |
| Lumiracoxib | 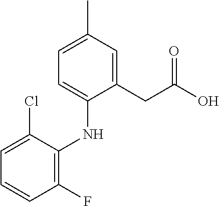 [SMILES: Cc1ccc(Nc2c(F)cccc2Cl)c(CC(=O)O)c1] |
| Amoxicillin-Clavulanate | 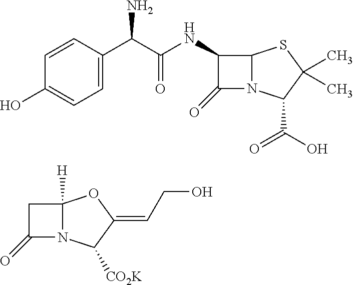 |
| Vancomycin | 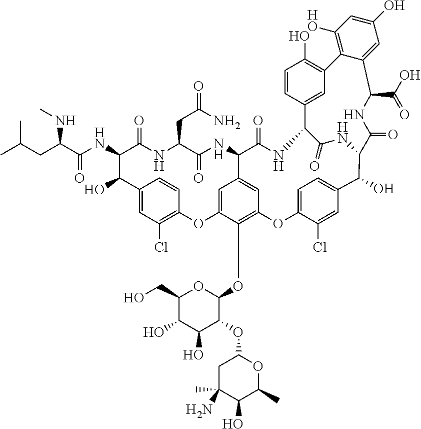 |
| Terbinafine | 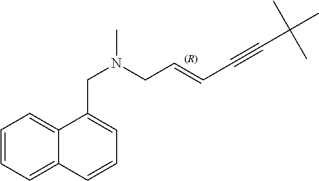 |
| Dapsone/nitroso Dapsone | 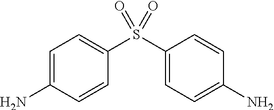 [SMILES: Nc1ccc(S(=O)(=O)c2ccc(N)cc2)cc1] |
| Raltegravir | 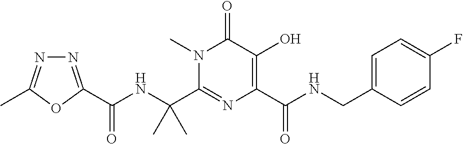 [SMILES: Cc1nnc(C(=O)NC(C)(C)c2nc(C(=O)NCc3ccc(F)cc3)c(O)c(=O)n2C)o1] |
| Lamotrigine | 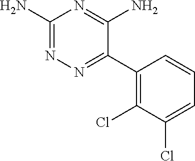 [SMILES: Nc1nnc(-c2cccc(Cl)c2Cl)c(N)n1] |
| “Cold Medicines” | Varying structures |
| Asparaginase | enzyme |
| Statins | Varying structures |

In some aspects, the present disclosure provides use of any one of the exemplary compounds described herein in the manufacture of a kit for use in the treatment of cancer. The present disclosure also provides use of any one of the exemplary compounds described herein as a medicament for use in the treatment of cancer. In some embodiments, the compound is an HLA binding molecule. In some embodiments, the use comprises administering a therapeutically effective amount of an HLA binding molecule to a subject. In some embodiments, the HLA binding molecule is a small molecule selected from the group consisting of small molecules listed in any one of Table 1, Table 2, FIGS. 4A-4I, and FIGS. 6A-6L. In some embodiments, the small molecule is abacavir or allopurinol.

In some embodiments, the use comprises a method comprising the steps (i) administering a first dose of abacavir or allopurinol sufficient to induce an immune response in the subject, and (ii) administering a second dose of abacavir or allopurinol. In some embodiments, the compound is a small molecule. In some embodiments, the use comprises administering a composition comprising a therapeutically effective amount of a small molecule to the subject, wherein the small molecule preferentially binds to one or more selective MHC allele and is capable of eliciting an immune hypersensitivity reaction in the subject.

### EXAMPLES

### Example 1: In Vitro Validation of Compound Selection Method in a Mouse System Targeting Hepatocellular Carcinoma (HCC)

Previous studies (see, e.g., Lucas, et al., Abacavir-Reactive Memory T Cells Are Present in Drug Naïve Individuals, PLoS One (2015); Ostrov, et al., Drug hypersensitivity caused by alteration of the MHC-presented self-peptide repertoire, Proc. Natl. Acad. Sci. U.S.A. (2012)), have shown that the FDA approved drug, abacavir, enhances CD8+ T cell activity by binding a specific HLA isotype: HLA-B*57:01. According to aspects of the methods described in the application, HLA-binding small molecule drugs can be used to enhance anti-tumor cytotoxic activity and treat cancer. By binding HLA, the drug alters the shape of the receptor in a manner that alters peptide binding, thus presenting neoantigen peptides recognized as targets for destruction by responding T cells.

HLA associations between adverse drug (e.g., small molecule) reactions and specific HLA alleles illustrate a set of approved drugs that enhance T cell responses in individuals carrying specific HLA alleles. Table 2 lists several examples of such FDA-approved drugs. Any of these drugs may be used in conjunction with the methods of treatment provided herein. As such, HLA binding drugs have the potential to boost immune responses in cancer patients. For example, cancer patients expressing HLA-B*57:01 may benefit from drugs such as abacavir.

Structural features common to at-risk HLA alleles were analyzed. In cancer patients expressing HLA-B, an improved response was found if the allele had a Y at position 9 and an L at position 156. Accordingly, a patient expressing this allele may benefit from an HLA binding drug to stimulate T cell responses against cancer.

TABLE 2
HLA associations between adverse drug
reactions and specific HLA alleles.

| Drug (e.g., small molecule) | Association(s) |
|---|---|
| Abacavir | B*57:01 |
| Allopurinol/Oxypurinol | B*58:01 |
| Carbamazepine | B*15:02 |
| | A*31:01 |
| Feprazone | B22 |
| Flucloxacillin | B*57:01 |
| Sulfamethoxazole | A30; B13; Cw6 |
| Sulfonamides (drug class) | A29; B12; DR7 |
| Levamisole | B27 |
| Oxicam | A2; B12 |
| Phenytoin | B*15:02 |
| Aspirin | DPB1*03:01 |
| | DRB1*13:02-DQB1*06:09 |
| Hydralazine | DR4 |
| Lapatinib | DRB1*07:01-DQA1* |
| | 02:01-DQB1*02:02/02:03 |
| NSAIDs (drug class) | DR11 |
| Ximelagatran | DRB1*07, DQA1*02 |
| | DQB1*07:01 |
| Aminopenicillins | A2, DRw52 |
| (drug class) | |
| Clozapine | B38, DR4, DR2 |
| d-penicillamine | B8, DR3, DR1 |
| Au+ Na+ Thiomalate | B8, DR3 |
| Nevirapine | Cw4-B14 |
| | B*35:05 |
| | Cw8 |
| | DRB1*01:01 |
| Methazolamide | A*59:01 |
| Ticlopine/Ticlopidine | A*33:03 |
| | DQB1*06:04 |
| Lumiracoxib | DRB1*15:01-DQB1*06:02- |
| | DRB5*01:01-DQA1*01:02 |
| Amoxicillin-Clavulanate | A*02:01-B*07:02-DRB1* |
| | 15:01-DQB1*06:02 |
| | A*02:01-B*18:01 |
| Vancomycin | A*32:01 |
| Terbinafine | A*33:01 |
| Dapsone/nitroso Dapsone | B*13:01 |
| Raltegravir | B*53:01 |
| Lamotrigine | A*68:01 |
| | B*15:02 |
| “Cold Medicines” | A*02:06 |
| | B*44:03 |
| Asparaginase | DRB1*07:01 |
| Statins | DRB1*11:01 |

Since only a small subset of HLA molecules have demonstrated associations with drug-induced enhancement of T cell activity, a method was developed to identify one or more drugs and/or drug-like molecules able to bind each HLA molecule (e.g., capable of binding at least one of the currently identified 26,512 HLA alleles). This method was validated through the testing of compound activities, selected using atomic structures of HLA molecules, in vitro.

Prior to validation in human systems, a mouse system targeting hepatocellular carcinoma (HCC), the most common form of liver cancer, was used. IMEA is a murine HCC line of the H2^{d} haplotype, expressing the H2-D^{d}, H2-K^{d}, and H2-L^{d} allelic variants.

In a first set of experiments, drugs (e.g., small molecules) that bind IMEA MHC were selected from a database compiled using the in silico modeling and scoring methodology disclosed herein, and were tested for T cell stimulation and HCC killing in vitro. The crystal structure of H2-D^{d} was used as the basis for in silico selection of FDA-approved drugs predicted to bind the antigen binding cleft. DOCK6, a structure-based design program for early drug discovery (available for download at http://dock.compbio.ucsf.edu/) was used to screen 1,207 FDA approved small molecule drugs by parallel processing at the University of Florida High Performance Computing Center. An initial 1,207 FDA approved compounds were selected based upon their predicted ability to bind the HLA-A2 supertype, and thus their predicted efficacy across a wide swath of individuals. The top 40 scoring compounds were obtained from the National Cancer Institute Developmental Therapeutics Program.

IMEA cells were seeded in 24-well plates. At 30% confluence of IMEA cells, 0.5×10⁶ of BALB/c mouse splenocytes were added to half of the wells. The compounds under evaluation were incubated with IMEA cells in concentrations of 10 mM and 1 mM, and supernatant was subsequently collected. IMEA cells were observed to be killed in the following groups: Compound 739 (at concentrations of 10 mM and 1 mM), compound 63878 (at concentrations of 10 mM and 1 mM), compound 102816 (10 mM), compound 163039 (10 mM), and compound 241286 (10 mM).

In vitro screening and identification revealed that three FDA approved drugs stimulated H2-D^{d} T cells and resulted in enhanced killing of IMEA HCC: ribavirin, melphalan and vidaza.

### Example 2: Validating HLA-Binding Small Molecules that Enhance T Cell Activity in Humans

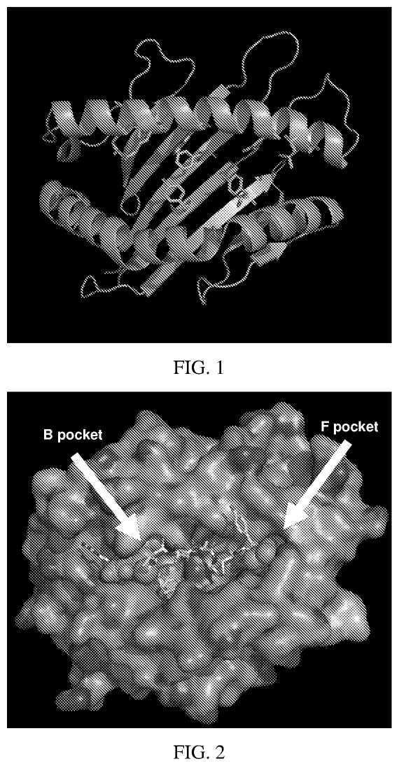

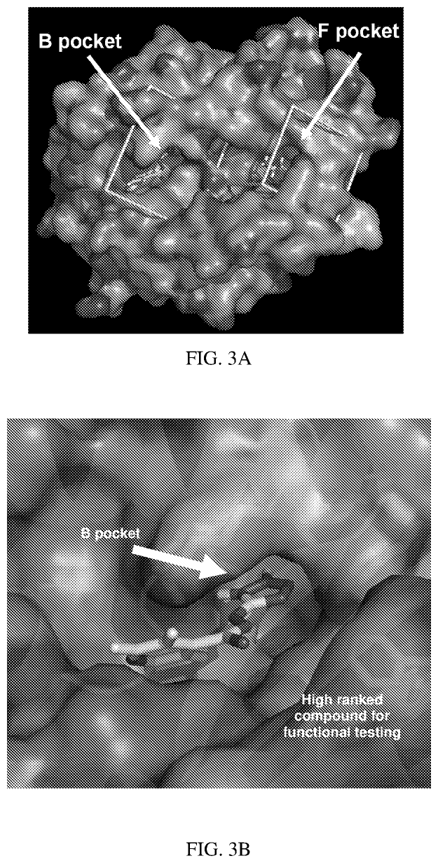

Next, a human system was used to validate HLA-binding small molecules that enhance T cell activity. The HLA-A2 allotype was selected as a promising target to enhance CD8⁺ T-cell responses because of its high frequency in human populations (27.2% in US Caucasians), which would allow the development of drug (e.g., small molecule) treatment for a large number of patients (FIG. 1). First, HLA supertype-specific pockets were successfully defined in the antigen binding cleft of HLA-A2 with structural features favorable for binding to drug-like small molecules (FIG. 2). Second, 1,207 FDA approved small molecule drugs were screened by in silico molecular docking to select candidates for functional T cell activity assays (FIGS. 3A and 3B). Using this approach, the top 40 or so scoring compounds were selected as candidates for ex vivo T cell stimulation assays.

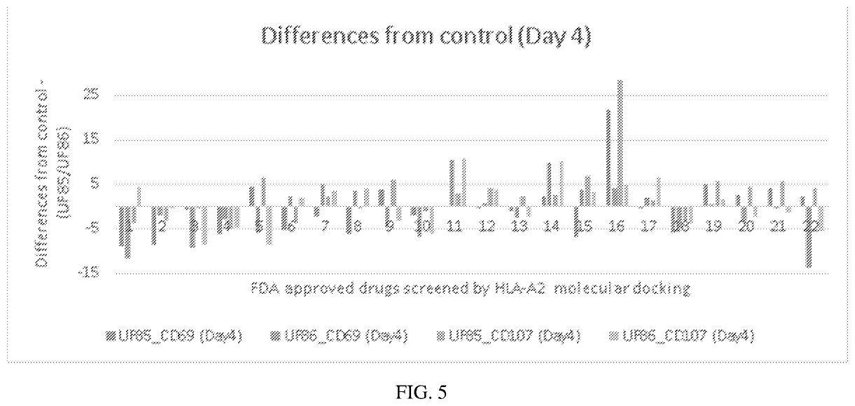

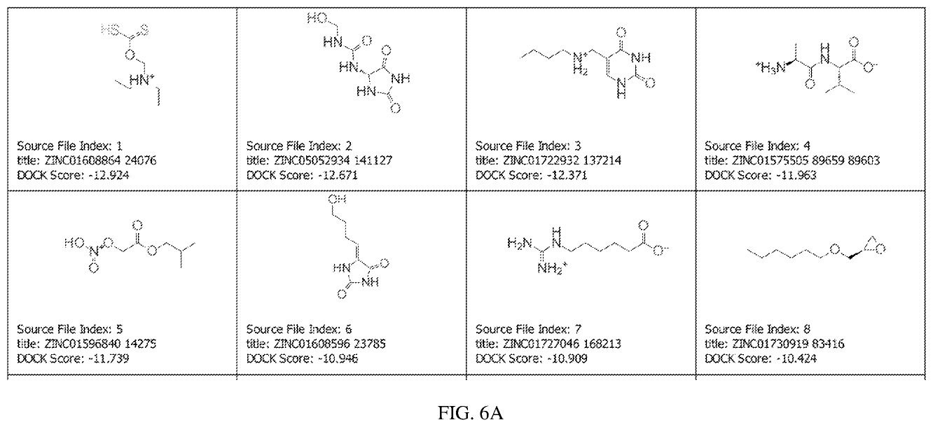

Third, top scoring compounds were tested for their ability to modulate proliferation of peripheral blood mononuclear cells (PBMCs) from healthy individuals using ex vivo T cell stimulation assays. Here, the drugs predicted to bind HLA-A2 (FIGS. 4A-4I) were tested using PBMCs from HLA-typed normal individuals. In assays, drugs were incubated with PBMCs (for up to 14 days), and functional characteristics of the responding T cell population were assayed utilizing flow cytometry (e.g., using antibodies specific for CD3 and CD8). PBMCs were stimulated with drugs at 10 mg per ml with interleukin-2 (IL-2) supplemented on day 2. FIG. 5 shows flow cytometric analysis of drug stimulated PBMCs on day 4. PBMCs from an HLA-A2 expressing individual (“UF 86” is homozygous for HLA-A*02:01) were incubated with a panel of drugs predicted to bind HLA-A2 by molecular docking. PBMCs from an individual lacking HLA-A2 (“UF 85”) served as a control for HLA allele specificity. Cells were stained with antibodies specific for CD3, CD8 (cytotoxic T lymphocyte markers), CD69, and CD107a (LAMP-1) (activation markers) to identify those drugs from the top scoring grouping that modify cytotoxic T lymphocyte activity.

No toxic effects at the concentration tested (50 μM) was observed for any of the tested compounds. Several compounds selected to bind HLA-A2 enhanced proliferation of HLA-A2⁺ PBMCs in a statistically significant manner. These data suggest that the HLA-based selection strategy and assays disclosed herein are useful for the development of functional CD8⁺ cell enhancement (flow cytometry and ELISPOT assays).

These data demonstrate that several FDA approved drugs modulate the activation of CD8 cells in an HLA-A2 homozygous individual (UF 86) without significant effects in an HLA-A2 negative individual (UF 85). For example, as shown in FIG. 5, drug 11 from (NSC 193417, 2,3-dibromo-4-oxobut-2-enoic acid) enhances the number of activated CD8⁺ cells in HLA-A2 expressing cells, but not HLA-A2 negative cells, presumably by altering the repertoire of peptides presented by HLA-A2. Compounds such as drug 11 would be candidates to consider for further study to identify the therapeutic index for future clinical trials.

Because HLA-A2 has been demonstrated to be associated with Graft-Versus-Host disease (GVHD), these data also demonstrate that there may be FDA approved drugs (e.g., small molecules) useful for prevention of GVHD by inhibiting HLA-A2 restricted T cell responses. For example, drugs 3 (NSC23842) and 10 (NSC109096) inhibit the autologous mixed lymphocyte reactions shown in FIG. 5 in the HLA-A2 expressing cells, but not control cells. These drugs may therefore inhibit T cell recognition using a mechanism wherein the drug hinders peptide/HLA-A2 interactions. These HLA-A2-specific immunosuppressive drugs represent candidates for further study to identify the therapeutic index for clinical trials.

Such studies also showed that Daltogen and Dagralax stimulate T cells from HLA-A2 expressing subjects but not from HLA-A2 negative subjects, indicating that Daltogen and Dagralax may be useful to treat disease (e.g., cancer) in HLA-A2 expressing subjects.

FIGS. 6A-6L show compounds selected to bind HLA-DR3.

### Example 3: A Clinical Trial for Evaluation of Abacavir Treatment of Cancer in HLA-B*57:01 Acute Myeloid Leukemia Patients

A clinical trial was designed to evaluate the effect of first and continued administrations of abacavir in subjects expressing HLA-B*57:01 to observe the effect of the drug (e.g., small molecule) on enhancement of T cell activity and validate the potential of the desired hypersensitivity reaction to neutralize a cancer. This trial was designed for subjects suffering from cancer, specifically acute myeloid leukemia (AML). Hypersensitivity reactions will be closely monitored by physicians during the trial.

The study is a phase 2 study in patients suffering from relapsed or refractory (R/R) acute myeloid leukemia (AML) or myelodysplastic syndromes (MDS) expressing the HLA-B*57:01 genotype. Despite complete remission rates of 40%-60% in older patients, even with best available therapy, only 5%-15% of older AML patients will have prolonged remissions or cures. Patients with relapse or refractory AML have very poor long-term survival, with median overall survival time of 6 months (Ganzel, et al., Am J Hematol. 2018 Jun. 15; 10.1002/ajh.25162). In MDS, only 40%-50% of patients achieve remission with a hypomethylating agent (HMA), and nearly all patients will suffer from relapsed disease. After failure of HMA, median survival time is 5.6 months (Prdbet, et al., J Clin Oncol. 2011 Aug. 20; 29(24):3322-7).

Allogeneic hematopoietic stem cell transplantation (HSCT) remains the treatment of choice in patients with AML or MDS who relapse, but many older patients are unable to achieve a second remission, do not have suitable donors, and do not tolerate the side effects of allogeneic HSCT such as organ toxicities, opportunistic infections, and graft versus host disease. Despite recent advances in the treatment of relapsed AML or MDS through the use of targeted biologic agents, most new agents produce relatively small numbers of complete responses lasting on the order of months.

Thus, there remains a large unmet medical need for novel treatments in patients with AML and MDS, especially those who are older or who are not candidates for allogeneic HSCT. Evidence shows that abacavir, an anti-viral agent approved for use in HIV patients, stimulates polyclonal T cell responses in drug naïve individuals that carry the HLA-B*57:01 allele (Lucas, et al., PLoS One 2015 Feb. 12; 10(2):e0117160; Bell, et al., Chem Res Toxicol. 2013 May 20; 26(5):759-66). Abacavir stimulates CD8 T cells that drive a systemic hypersensitivity syndrome in individuals that carry the HLA-B*57:01 allele by a well-characterized mechanism (Ostrov, et al., Proc Natl Acad Sci USA. 2012 Jun. 19; 109(25):9959-64). It is expected that eliciting a systemic hypersensitivity reaction by enhancing adaptive autologous adaptive immunity with abacavir in AML and MDS patients with HLA-B*57:01 genotype will sensitize the patient's own immune system against their malignant cells leading to disease regression.

Subjects will receive abacavir 600 mg by mouth daily every day during 28-day cycles. Dose reductions to 400 mg PO per day will be permitted if the subject has or develops mild hepatic impairment (Child-Pugh class A). Treatment continues until disease progression or drug intolerance. Additional details of the Phase 2 study follow:

TABLE 3
Study Design and Objectives.

| | |
|---|---|
| Title | A Phase 2 Study of Abacavir in |
| | Relapsed or Refractory Acute |
| | Myeloid Leukemia or Myelodysplastic Syndromes |
| Study Phase | 2 |
| Study Sites | 1 |
| Indication | Relapsed or refractory (R/R) acute |
| | myeloid leukemia (AML) or |
| | myelodysplastic syndromes (MDS) with |
| | HLA-B*57:01 genotype |
| Rationale | Despite complete remission rates of |
| | 40%-60% in older patients, even |
| | with best available therapy, only 5%-15% |
| | of older AML patients will |
| | have prolonged remissions or cures. |
| | Patients with relapse or refractory |
| | AML have very poor long-term survival, |
| | with median overall survival |
| | time of 6 months (Ganzel, et al., |
| | Am J Hematol. 2018 Jun |
| | 15; 10.1002/ajh.25162). In MDS, |
| | only 40%-50% of patients achieve |
| | remission with a hypomethylating agent |
| | (HMA), and nearly all patients |
| | will suffer from relapsed disease. After |
| | failure of HMA, median survival |
| | time is 5.6 months (Prébet, et al., J Clin Oncol. |
| | 2011 Aug 20; 29(24): 3322-7). |
| | Allogeneic hematopoietic stem cell |
| | transplantation (HSCT) remains the |
| | treatment of choice in patients with AML |
| | or MDS who relapse, but many |
| | older patients are unable to achieve a |
| | second remission, do not have |
| | suitable donors, and do not tolerate the |
| | side effects of allogeneic HSCT |
| | such as organ toxicities, opportunistic |
| | infections, and graft versus host |
| | disease. Despite recent advances in the |
| | treatment of relapsed AML or |
| | MDS through the use of targeted biologic |
| | agents, most new agents |
| | produce relatively small numbers of |
| | complete responses lasting on the |
| | order of months. |
| | Thus, there remains a large unmet medical |
| | need for novel treatments |
| | in patients with AML and MDS, especially |
| | those who are older or who |
| | are not candidates for allogeneic HSCT. |
| | Evidence show that abacavir, an anti-viral |
| | agent approved for use in |
| | HIV patients, stimulates polyclonal T cell |
| | responses in drug naïve individuals that |
| | carry the HLA-B*57:01 allele (Lucas, et al., PLoS One |
| | 2015 Feb 12; 10(2): e0117160; Bell, et al., |
| | Chem Res Toxicol. 2013 |
| | May 20; 26(5): 759-66). Abacavir stimulates |
| | CD8 T cells that drive a systemic |
| | hypersensitivity syndrome in individuals that |
| | carry the HLA-B*57:01 allele by a |
| | well characterized mechanism (Ostrov, et al., Proc |
| | Natl Acad Sci U S A. 2012 Jun 19; 109(25): 9959-64). |
| | We expect that eliciting a systemic hypersensitivity |
| | reaction by enhancing adaptive autologous |
| | adaptive immunity with abacavir in |
| | AML and MDS patients with HLA-B*57:01 |
| | genotype will sensitize the patient's own |
| | immune system against their malignant cells |
| | leading to disease regression. |
| Objectives | Primary Objective |
| | The primary objective of the trial is to compare |
| | the overall survival (OS) time of |
| | R/R AML or MDS patients treated with abacavir |
| | versus historical reference cohorts. |
| | Secondary Objectives |
| | • To assess the safety & tolerability of abacavir |
| | as measured by clinical reporting of |
| | adverse events, findings on physical exam |
| | and laboratory parameters in subjects with |
| | R/R AML or MDS. |
| | • To evaluate the efficacy of abacavir compared |
| | to reference cohorts with respect to: |
| | ○ OS rate (%) at 3, 6, 9 and 12 months |
| | ○ Progression Free Survival (PFS) time |
| | ○ PFS rate (%) at 3, 6, 9, and 12 months |
| | ○ Measurable Residual Disease (MRD) by multigene |
| | assay at 3, 6, 9, and 12 months |
| | Exploratory Objectives |
| | • To determine the antigen-specific (HLA-B*57:01) T-cell |
| | (CD8/CD4) immune-response in patients receiving |
| | abacavir in peripheral blood specimens |
| | • To determine associations of abacavir |
| | efficacy with degree |
| | of HLA-B*57:01 cell surface expression on malignant |
| | blasts. We expect that higher HLA-B*57:01 cell surface |
| | expression associates with greater disease regression. |
| | • To determine response associations with pathogen |
| | infected patients, e.g., human herpes viruses. |
| | • To determine response associations with other |
| | HLA and non-HLA genes. |
| | • To determine AML clonal evolution and tumor |
| | microenvironment molecular “signatures” in the bone |
| | marrow and peripheral blood using bone marrow |
| | aspirate and peripheral blood specimens |
| Study | This is an open-label study of abacavir in patients |
| Design | with R/R AML or MDS with HLA-B* |
| | 57:01 genotype. The primary goal of the study will |
| | be to demonstrate an advantage for abacavir in overall |
| | survival in these patient populations. |
| | The study will enroll approximately 13 patients and |
| | will be conducted at one site. |
| | Patients will receive 600 mg of abacavir by mouth |
| | daily every day of a 28-day cycle. |
| | Patients will be evaluated by clinical examination every |
| | week for a month and then every 4 weeks thereafter. |
| | All patients will undergo bone marrow aspirates |
| | and biopsies at Week 12. Bone marrow |
| | examinations will then be repeated as clinically |
| | indicated. Patients will be assessed for safety at each |
| | of the clinical examination visits. |
| Subject Key | Inclusion Criteria |
| Eligibility | 1. Patients, or their legally acceptable |
| Criteria | representatives, must be willing and able to |
| | understand and provide signed informed |
| | consent for the study that fulfills Institution |
| | Review Board (IRB) guidelines |
| | 2. Male or female patients ≥ 18 years of age on the |
| | day of signing informed consent |
| | 3. Subjects must have a diagnosis of AML according |
| | to the WHO criteria (primary/de novo |
| | or secondary, including treatment- |
| | related [e.g., due to prior anthracycline use], |
| | as well as cases due to progression of |
| | antecedent hematological disorder [e.g., MDS, MPN, |
| | or MDS/MPN ‘overlap’ syndrome), or a diagnosis of |
| | MDS according to WHO criteria (primary/de novo or |
| | secondary). |
| | 4. Subjects must have relapsed or refractory disease, |
| | defined as persistent AML or |
| | MDS despite at least one cycle of disease- |
| | modifying treatment. |
| | 5. Not eligible for high intensity reinduction |
| | chemotherapy, such as |
| | CLAM, CECA, FLAG, or MEC chemotherapy. |
| | 6. Subjects must have the HLA-B*57:01 genotype |
| | 7. Subjects must not be candidates at the time of |
| | study entry for allogeneic stem |
| | cell transplant (Allo-SCT) due to intercurrent |
| | medical conditions or lack of an available donor. |
| | 8. Subjects must have received the last dose of |
| | disease modifying therapy at least one |
| | month prior to expected first day of abacavir |
| | treatment. |
| | 9. Subjects must have an Eastern Cooperative Oncology |
| | Group (ECOG) performance status of 0, 1 or 2. |
| | 10. Subjects must have an estimated life |
| | expectancy > 3 months. |
| | 11. If female, is postmenopausal (at least 12 sequential |
| | months of amenorrhea) or surgically sterile. |
| | 12. Females of childbearing potential must have a negative |
| | pregnancy test |
| | 13. Female patients of childbearing potential |
| | who are heterosexually |
| | active and male patients with female sexual partners of |
| | childbearing potential must agree to use an |
| | effective method of contraception (e.g., |
| | oral contraceptives, double-barrier methods |
| | such as a condom and a diaphragm, |
| | intrauterine device) during the study |
| | and for 4 months following the last dose of study |
| | medication, or to abstain from sexual intercourse |
| | for this time; a woman not of |
| | childbearing potential is one who has undergone |
| | bilateral oophorectomies or who is post-menopausal, |
| | defined as the absence of menstrual |
| | periods for 12 consecutive months. |
| | 14. Subjects must have adequate renal function |
| | defined as a serum creatinine < 2 × upper |
| | limit of normal (ULN) or calculated |
| | creatinine clearance ≥ 30 mL/min based on the |
| | Cockroft-Gault equation. |
| | 15. Subjects must have adequate hepatic function |
| | defined as a serum total bilirubin < 2 × |
| | ULN (except for Gilbert's syndrome, which will |
| | allow bilirubin ≤ 3.0 mg/dL), and alanine |
| | aminotransferase (ALT) and |
| | aspartate aminotransferase (AST) ≤ 3 × ULN. |
| | 16. Subjects must be willing and able to return |
| | to the clinical site for adequate follow-up |
| | and to comply with the protocol as required. |
| | Exclusion Criteria |
| | 1. Subjects with an imminently planned hematopoietic |
| | stem cell transplant (autologous |
| | or allogeneic, with any degree of match donor). |
| | 2. Subjects with acute promyelocytic leukemia or any |
| | morphologic and molecular variants, inclusive. |
| | 3. Subjects with a serious concurrent illness that |
| | in the opinion of the |
| | Investigator would pose an undue risk to the subject being |
| | participating in the clinical study. |
| | 4. Subjects with a history of, or who currently have, central |
| | nervous system leukemia. |
| | 5. Is currently participating in or has participated in a |
| | study of an investigational agent or |
| | has used an investigational device within 4 |
| | weeks prior to the first dose of study treatment. |
| | 6. Has a diagnosis of immunodeficiency or is |
| | receiving chronic systemic steroid therapy |
| | (in dosing exceeding 10 mg daily of |
| | prednisone equivalent) or any other form of |
| | immunosuppressive therapy within 7 days |
| | prior the first dose of study drug. The use of |
| | physiologic doses of corticosteroids may be approved after |
| | consultation with the Sponsor. Steroids taken as short-term |
| | therapy (≤7 days) for antiemesis are permissible. |
| | 7. Has a known additional malignancy that is progressing |
| | or has required active treatment |
| | within the past 5 years, even if currently |
| | inactive or unapparent. |
| | 8. Has an active autoimmune disease that has |
| | required systemic treatment |
| | in past 2 years (i.e., with use of disease modifying |
| | agents, corticosteroids or immunosuppressive drugs). |
| | Replacement therapy (e.g., thyroxine, insulin, |
| | or physiologic |
| | corticosteroid replacement therapy for adrenal or pituitary |
| | insufficiency) is not considered a form of systemic |
| | treatment and is allowed. |
| | 9. Has an active and uncontrolled infection requiring |
| | systemic therapy. |
| | 10. Has a history or current evidence of any condition, |
| | therapy, or laboratory abnormality that |
| | might confound the results of the study, |
| | interfere with the patient's participation for the full |
| | duration of the study, or is not in the |
| | best interest of the participant to participate, |
| | in the opinion of the treating investigator. This includes any |
| | serious, intercurrent, chronic, or acute illness, such as |
| | cardiac disease (New York Heart |
| | Association [NYHA] class III or IV), |
| | hepatic disease, or other illness considered by the |
| | investigator as an unwarranted high |
| | risk for investigational drug treatment.20. Has a |
| | known psychiatric or substance abuse disorder that would |
| | interfere with the participant's |
| | ability to cooperate with the requirements of the study. |
| | 11. Is pregnant or breastfeeding or expecting to conceive or |
| | father children within the projected |
| | duration of the study, starting with the |
| | screening visit through 30 days after the last dose of study |
| | treatment. |
| | 12. Has had an allogeneic hematopoietic or |
| | solid organ transplant. |
| Study | Abacavir: |
| Treatments | Subjects will receive abacavir 600 mg by mouth |
| | daily every day during 28-day cycles. |
| | Dose reductions to 400 mg PO QD will be |
| | permitted if the subject has or develops mild hepatic |
| | impairment (Child-Pugh class A). |
| | Treatment continues until disease progression |
| | or drug intolerance. |
| Statistical | Primary Efficacy Endpoint |
| Analysis | The primary efficacy endpoint is overall survival time. |
| | Secondary Efficacy Endpoints |
| | The secondary efficacy endpoints are OS rate at 3, 6, 9, |
| | and 12 months; Progression Free |
| | Survival (PFS) time; PFS rates at 3, 6, 9, and 12 |
| | months; and presence of MRD at 3, 6, 9, and 12 months. |
| | Exploratory Endpoints |
| | The exploratory endpoints are HLA-B*57:01 specific |
| | immune response dynamics in PB |
| | and select general immunodynamics |
| | assessments in PB and bone marrow. |
| | Sample Size Calculations |
| | The historical reference cohort for R/R AML patients |
| | will be the Ganzel, et al cohort who |
| | demonstrated a median OS time of 6 months |
| | after AML relapse (Ganzel, et al., Am J Hematol. 2018 Jun |
| | 15; 10.1002/ajh.25162). The historical reference cohort |
| | for R/R MDS patients will be the Prébet, |
| | et al cohort who had a median survival |
| | Overall Survival time of 5.6 months after failing azacitidine |
| | chemotherapy (Prébet, et al., J Clin Oncol. 2011 |
| | Aug 20; 29(24): 3322-7). |
| | A total of 13 subjects will be enrolled in the study, which |
| | will provide at least 90% power under an |
| | assumed hazard ratio (HR) of 0.66, based on a median |
| | OS of 6 months in the reference cohort versus an expected |
| | OS of 9 months in the study cohort, and a standard |
| | deviation of 2 months. |
| | Analysis Sets |
| | All treated subjects will constitute the full analysis set |
| | (FAS). All subjects who receive at |
| | least one dose of study treatment, and do not |
| | deviate from the protocol in any major way will constitute |
| | the modified intention-to-treat |
| | (mITT) set. All subjects who receive 8 weeks of |
| | treatment, and do not deviate from the protocol in any |
| | major way will constitute the per-protocol |
| | set (PPS). All efficacy analyses will be |
| | based on either the FAS or the mITT set. |
| | Exploratory analyses will be performed on the PPS set. |
| | All patients who receive at least one dose of treatment will |
| | be included in the safety analysis (SA) set. |
| | The safety analyses will be based on the SA set. |
| | Further details will be described in the study Statistical |
| | Analysis Plan (SAP). |
| | Statistical Analysis Methodology Summary |
| | The primary efficacy analysis will be conducted in the FAS |
| | by using a Cox proportional hazards |
| | model, and with treatment as the only |
| | independent variable, to estimate the hazard ratio (HR) of |
| | the abacavir cohort versus the |
| | reference cohort, and to test the null hypothesis H0: |
| | HR ≥ 1 versus the alternative hypothesis H1: HR < |
| | 1. Testing will be conducted at an overall one-sided |
| | significance level of 0.025. |
| | Continuous variables will be summarized by a clinically |
| | relevant discretization, as appropriate. Multiple safety and |
| | demographic data will be summarized using standard |
| | tabulations and listings. Continuous variables will be |
| | summarized using mean, standard deviation, median, |
| | minimum value, and maximum value. |
| | Categorical variables will be summarized using frequency |
| | counts and percentages. Data listings will be provided. |
| | Time to event data will be summarized using the Kaplan- |
| | Meier method. Where appropriate, 95% confidence |
| | intervals around point estimates will be |
| | presented, and estimates of the median and other |
| | quantiles, as well as individual time points |
| | (e.g. 3-month, 6-month, 9-month, and 12-month rates), |
| | will be produced. |
| | Further details will be described in the study |
| | Statistical Analysis Plan (SAP). |

Table 4 shows predicted white blood cell count, absolute lymphocyte count, and absolute T cell count upon administration of abacavir to various patient populations according to the protocol shown in Table 3.

TABLE 4
Predicted white blood cell, lymphocyte, and T cells counts in subjects
administered abacavir according to the protocol described in Table 3.

| | White | Absolute | |
|---|---|---|---|
| Description of | Blood | Lympho- | Absolute |
| subject receiving | Cell | cyte | T Cell |
| abacavir treatment | Count | Count | Count |
| Non-cancer patient who is not HLA- | 4.5 to | 1 to 4 × | 0.5 to 1.6 × |
| B*57:01⁺ and treated with abacavir | 11 × 10⁹/L | 10⁹/L | 10⁹/L |
| Non-cancer patient who is HLA- | 4.5 to | 1 to 4 × | 0.5 to 1.6 × |
| B*57:01⁺, treated with abacavir, and | 11 × 10⁹/L | 10⁹/L | 10⁹/L |
| has no hypersensitivity reaction | | | |
| Non-cancer patient who is HLA- | 6.75 to | 1.5 to 6 × | 0.75 to |
| B*57:01⁺, treated with abacavir, and | 16.5 × | 10⁹/L | 2.4 × 10⁹/L |
| has mild hypersensitivity reaction | 10⁹/L | | |
| Non-cancer patient who is HLA- | 9 to 22 × | 2 to 8 × | 1 to 3.2 × |
| B*57:01⁺, treated with abacavir, and | 10⁹/L | 10⁹/L | 10⁹/L |
| has moderate hypersensitivity reaction | | | |
| Non-cancer patient who is HLA- | 13.5 to | 3 to 12 × | 1.5 to 4.8 × |
| B*57:01⁺, treated with abacavir, and | 33 × 10⁹/L | 10⁹/L | 10⁹/L |
| has severe hypersensitivity reaction | | | |
| Cancer patient who is not HLA- | 4.5 to | 1 to 4 × | 0.5 to 1.6 × |
| B*57:01⁺ and treated with abacavir | 11 × 10⁹/L | 10⁹/L | 10⁹/L |
| Cancer patient who is HLA-B*57:01⁺, | 4.5 to | 1 to 4 × | 0.5 to 1.6 × |
| on no chemotherapy or biotherapy, | 11 × 10⁹/L | 10⁹/L | 10⁹/L |
| treated with abacavir, and has no | | | |
| hypersensitivity reaction | | | |
| Cancer patient who is HLA-B*57:01⁺, | 6.75 to | 1.5 to 6 × | 0.75 to |
| on no chemotherapy or biotherapy, | 16.5 × | 10⁹/L | 2.4 × 10⁹/L |
| treated with abacavir, and has mild | 10⁹/L | | |
| hypersensitivity reaction | | | |
| Cancer patient who is HLA-B*57:01⁺, | 9 to 22 × | 2 to 8 × | 1 to 3.2 × |
| on no chemotherapy or biotherapy, | 10⁹/L | 10⁹/L | 10⁹/L |
| treated with abacavir, and has | | | |
| moderate hypersensitivity reaction | | | |
| Cancer patient who is HLA-B*57:01⁺, | 13.5 to | 3 to 12 × | 1.5 to 4.8 × |
| on no chemotherapy or biotherapy, | 33 × 10⁹/L | 10⁹/L | 10⁹/L |
| treated with abacavir, and has severe | | | |
| hypersensitivity reaction | | | |
| Cancer patient who is HLA-B*57:01⁺, | 2 to 8 × | 0.5 to 2 × | 0.25 to |
| on chemotherapy or biotherapy, | 10⁹/L | 10⁹/L | 1 × 10⁹/L |
| treated with abacavir, and has no | | | |
| hypersensitivity reaction | | | |
| Cancer patient who is HLA-B*57:01⁺, | 4 to 11 × | 1 to 4 × | 0.5 to 2 × |
| on chemotherapy or biotherapy, | 10⁹/L | 10⁹/L | 10⁹/L |
| treated with abacavir, and has mild | | | |
| hypersensitivity reaction | | | |
| Cancer patient who is HLA-B*57:01⁺, | 6 to 24 × | 1.5 to 6 × | 0.75 to |
| on chemotherapy or biotherapy, | 10⁹/L | 10⁹/L | 3 × 10⁹/L |
| treated with abacavir, and has | | | |
| moderate hypersensitivity reaction | | | |
| Cancer patient who is HLA-B*57:01⁺, | 8 to 24 × | 2 to 8 × | 1 to 4 × |
| on chemotherapy or biotherapy, | 10⁹/L | 10⁹/L | 10⁹/L |
| treated with abacavir, and has severe | | | |
| hypersensitivity reaction | | | |

### Example 4: Assays for Immune Cell Activation and/or Tumor Cell Killing Induced by Drugs in an MHC Allele Specific-Manner

To assess the ability of drugs, especially drugs approved by FDA, to drive immune cell activation and/or tumor cell killing in an MHC allele-specific manner, the following experiment is performed.

Cryopreserved human peripheral blood mononuclear cells (hPBMCs) from donors that are positive for a specific MHC allele are thawed and labeled with Tag-It-Violet (Biolegend #425101), a proliferation dye. hPBMCs from donors that are negative for the desired MHC allele are included as controls. Labeled hPBMCs are cultured with or without a human tumor cell line that also expresses the desired MHC allele at a 10:1 ratio in a 96-well plate. The cell samples in the 96-well plate are treated with or without a drug of interest at various concentrations. After approximately 72 hours, plates are spun, and supernatants are collected and frozen for future cytokine analysis as an indication of immune cell activation. To further assess immune cell activation, the immune cells are then stained for activation markers CD25 and CD69 utilizing respective commercially available antibodies, such as Biolegend #302606 and Biolegend #310932, in the presence of an Fc receptor blocking agent. Stained cells are fixed prior to analysis on a Cytek spectral flow cytometer. Tumor cell killing can be established by staining the tumor cells with a commercially available viability dye.

### Other Embodiments

It should be understood that the examples and embodiments described herein are for illustrative purposes only and that various modifications or changes in light thereof will be suggested to persons skilled in the art and are to be included within the spirit and purview of this disclosure and the scope of the appended claims.

All references cited herein (including publications, patent applications and patents) are incorporated by reference to the same extent as if each reference was individually and specifically incorporated by reference, and was set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order, unless otherwise indicated herein, or unless otherwise clearly contradicted by context.

The use of any examples, or exemplary language (e.g., “such as”) provided herein, is intended merely to better illustrate the disclosure and does not pose a limitation on the scope of the disclosure unless otherwise indicated. No language in the specification should be construed as indicating any element is essential to the practice of the disclosure unless as much is explicitly stated.

The description herein of any aspect or embodiment of the disclosure using terms such as “comprising”, “having”, “including” or “containing” with reference to an element or elements is intended to provide support for a similar aspect or embodiment of the disclosure that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and/or methods disclosed herein, and/or to the steps or the sequence of steps of the methods described herein without departing from the concept, spirit and/or scope of the disclosure. More specifically, it will be apparent that certain agents that are chemically- and/or physiologically-related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method comprising administering a therapeutically effective amount of an HLA binding molecule to a subject, wherein the HLA binding molecule is a small molecule selected from the group consisting of small molecules listed in any one of Table 1, Table 2, FIGS. 4A-4I, and FIGS. 6A-6L.

2. The method of claim 1, wherein the HLA binding molecule is a small molecule selected from the group consisting of small molecules listed in any one of Table 1.

3. The method of claim 1, wherein the HLA binding molecule binds to HLA-B.

4. The method of claim 1, wherein the HLA binding molecule binds to HLA-DR.

5. The method of claim 1, wherein the HLA binding molecule binds to HLA-A2.

6. The method of claim 3, wherein the HLA binding molecule is Abacavir, Allopurinol, or a combination thereof.

7. The method of claim 4, wherein the HLA binding molecule is selected from the group of molecules listed in FIGS. 6A-6L.

8. The method of claim 5, wherein the HLA binding molecule is Daltogen, Dagralax, or a combination thereof.

9. The method of any preceding claim, wherein the subject is a human subject.

10. The method of any preceding claim, wherein the subject has been diagnosed as having a cancer.

11. The method of claim 10, wherein the cancer is selected from lung, liver, pancreatic, stomach, colon, brain, breast, skin, or other cancer.

12. The method of any preceding claim, wherein the therapeutically effective amount slows the development, growth, or spread of a cancer.

13. The method of any preceding claim, wherein the therapeutically effective amount kills one or more cancer cells.

14. The method of any preceding claim, further comprising administering one or more additional anti-cancer drugs to the subject.

15. The method of any preceding claim, further comprising administering a cancer specific antigen to the subject.

16. A method of identifying a compound that enhances T cell mediated immunity by HLA binding, the method comprising:
i) performing a structure-based analysis to identify a compound that binds to an HLA molecule, and
ii) evaluating the compound identified in a) using a cell-based assay and/or an animal model to determine whether the compound enhances T cell mediated immunity.

17. The method of claim 17, wherein step (a) comprises a step of modeling in silico the structure of the HLA allele of interest.

18. A method of treatment of a subject suffering from or diagnosed with cancer comprising:
i) administering a first dose of abacavir or allopurinol sufficient to induce an immune response in the subject, and
ii) administering a second dose of abacavir or allopurinol.

19. The method of claim 18, further comprising administering a third dose of abacavir or allopurinol.

20. The method of claim 18 or 19, wherein the first dose, second dose, and/or third dose is 100 mg/day, 200 mg/day, 300 mg/day, 400 mg/day, 500 mg/day, or 600 mg/day.

21. The method of any one of claims 18-20, wherein the first dose, second dose, and/or third dose is 400 mg/day.

22. The method of any one of claims 18-20, wherein the first dose, second dose, and/or third dose is 600 mg/day.

23. The method of any one of claims 18-22, wherein the second dose is higher than the first dose.

24. The method of any one of claims 18-23, wherein the steps of administering provide amelioration of symptoms associated with the cancer.

25. The method of any one of claims 18-24, wherein the method provides improvement in any of the following endpoints: overall survival (OS) rate, OS time, Progression Free Survival (PFS) time, PFS rate, and Measurable Residual Disease (MRD).

26. The method of any one of claims 18-25, wherein the method provides improvement in any of the following endpoints: morphologic remission rate, time to achieve morphologic remission, cytogenetic remission rate, time to achieve cytogenetic remission, molecular remission rate, time to achieve molecular remission, progression free survival rate, and progression free survival time.

27. The method of claim 25 or 26, wherein the endpoint is assessed at 1, 2, 3, 6, 9, 12, 15, 18, and/or 24 months after the administering of a first dose of abacavir or allopurinol.

28. The method of any one of claims 18-27, wherein the steps of administering slows the development, progression, and/or spread of a cancer in the subject.

29. A method of augmenting an anti-cancer or anti-tumor immune response in a subject in need thereof, comprising: administering a composition comprising a therapeutically effective amount of a small molecule to the subject, wherein the small molecule preferentially binds to one or more selective MHC allele and is capable of eliciting an immune hypersensitivity reaction in the subject, thereby augmenting said anti-cancer or anti-tumor immune response.

30. The method of claim 29, wherein the small molecule preferentially binds to one or more selective HLA and elicits the immune hypersensitivity reaction in the subject, thereby augmenting said anti-cancer or anti-tumor immune response.

31. The method of claim 29, wherein the small molecule is a molecule selected from the group consisting of Table 1, Table 2, FIGS. 4A-4I, and FIGS. 6A-6L.

32. The method of claim 29, wherein the small molecule is abacavir or allopurinol.

33. The method of claim 29, wherein the selective MHC allele is class I.

34. The method of claim 29, wherein the selective MHC allele is class II.

35. The method of claim 30, wherein the HLA is HLA-B*57 or HLA-B*58.

36. The method of claim 30, wherein the small molecule elicits the immune hypersensitivity reaction in the subject that expresses the selective HLA.

37. The method of claim 29, wherein the composition comprises abacavir and allopurinol.

38. The method of claim 29, wherein the step of administering takes place in conjunction with another therapy.

39. The method of claim 38, wherein the step of administering takes place before, after or concurrently with the another therapy, wherein the another therapy is selected from the group consisting of: a chemotherapy, a cell therapy, an antibody therapy, and a combination thereof.

40. The method of any one of claims 29-39, wherein the therapeutically effective amount of a small molecule administered to the subject is less than an amount capable of causing the immune hypersensitivity reaction in said subject.

41. The method of any one of claims 29-40, wherein the subject exhibits a propensity for immune hypersensitivity elicited by the small molecule.

42. The method of claim 41, wherein the propensity for immune hypersensitivity is ascertained by testing for the presence of the one or more selective MHC to which the small molecule binds.

43. A method of treatment of a subject suffering from or diagnosed with cancer comprising:
i) administering a first dose of abacavir sufficient to induce an immune response in the subject, and
ii) administering a second dose of abacavir.

44. A method comprising:
i) administering orally a first dose of abacavir to a subject suffering from or diagnosed with cancer, and
ii) administering orally a second dose of abacavir to the subject,
wherein the subject has an HLA-B*57:01 genotype.
